(19) United States
(12) Patent Application Publication
(10) Pub. No.: US 2011/0144079 A1
(43) Pub. Date: Jun. 16, 2011

(54) ANTIMICROBIAL SYSTEM

(76) Inventors: Martyn Earle, Belfast (GB); Ken Seddon, Belfast (GB); Manuela Gilea, Belfast (GB); Gabor Boberly, Belfast (GB); Brendan Gilmore, Belfast (GB); Sean Gorman, Belfast (GB); Martin McLaughlin, Belfast (GB)

(21) Appl. No.: 12/937,149

(22) PCT Filed: Apr. 8, 2009
(86) PCT No.: PCT/GB09/50343

§ 371 Date: Mar. 2, 2011

(30) Foreign Application Priority Data
Apr. 11, 2008 (GB) 0806608.6
Oct. 24, 2008 (GB) 0819599.2

(51) Int. Cl. A61K 31/555; C07F 1/10; C07F 1/08; C07F 7/22; C07F 3/06; A01N 55/04; A01N 55/02; A01P 1/00

(52) U.S. Cl. 514/187; 548/101; 546/11; 546/2; 544/64; 544/226; 544/225; 546/10; 514/188; 514/189; 514/184

(57) ABSTRACT

The present invention relates to the use of ionic liquids as antimicrobial agents, and to the use of antimicrobial compositions comprising an ionic liquid, a film-enhancing composition and a viscosity controlling agent. The invention further relates to methods of disinfecting substrate surfaces, to substrates produced by such methods, and to novel ionic liquid compositions.

The present invention is directed to an antimicrobial system, and more specifically to an antimicrobial system which is comprised of a film-enhancing composition, a viscosity controlling agent and an ionic liquid. The present invention is also directed to uses of such compositions, to methods of disinfecting substrate surfaces, and to substrates comprising an antimicrobial layer. The invention is further directed to novel ionic liquid compositions and their use as antimicrobial agents.

Pathogenic microbes pose a significant threat to human health, and a variety of solutions have been developed to counter this threat. A particular area of concern is the microbial contamination of surfaces and the potential for the spread of disease and infection by contact with such contaminated surfaces. Effective disinfecting regimes are necessary to reduce microbial contamination of susceptible surfaces, for example in domestic and health care environments. In particular, effective disinfecting solutions are an important measure in preventing the spread of hospital-acquired infections, such as those attributable to pathogens such as methicillin-resistant S. aureus (MRSA), C. difficile, H. Pylori, Salmonella and E. coli.

Typically, disinfecting measures kill fungi and/or bacteria that are present on surfaces at the time that they are applied, but tend to do so effectively only at the time of application. One reason for this is that the majority of disinfecting agents, once dried through evaporation, provide no protection against future infection of the surface. In addition, general cleaning, such as wiping a surface with a cloth, can remove many known disinfecting agents. Such surfaces can easily suffer recontamination, requiring frequent reapplication of the disinfectant. Furthermore, conventional disinfectant solutions often have to be applied in relatively high concentrations in order to obtain broad spectrum disinfection. High concentrations of disinfectants are hazardous if brought into contact with food, and may also cause skin and eye irritation.

There is therefore a need for novel disinfecting compositions which provide broad spectrum antimicrobial activity over prolonged periods of time. Preferably such compositions comprise an active antimicrobial agent at levels that do not pose toxicity problems to humans and animals.

Ammonium and phosphonium compounds have been suggested for use as antimicrobial compounds against gram-positive and gram-negative bacteria, fungi, protozoa and certain viruses.

The inventors of the present invention have surprisingly found that ionic liquids comprising or consisting of heterocyclic cations have superior wide-spectrum antimicrobial activity compared to known ammonium and phosphonium compounds which have hitherto been described in the prior art. In particular, the ionic liquids of the present invention have been found to have increased antimicrobial activity when compared to the industry standard disinfectant benzalkonium chloride (alkyldimethylbenzylammonium chloride). The increased antimicrobial activity potentially allows for the amount of disinfectant used to be reduced with a corresponding reduction in toxicity.

Ionic liquids are a novel class of compounds which have been developed over the last few years. The term “ionic liquid” as used herein refers to a liquid that is capable of being produced by melting a solid, and when so produced consists solely of ions. Ionic liquids may be derived from organic salts.

An ionic liquid may be formed from a homogeneous substance comprising one species of cation and one species of anion, or it can be composed of more than one species of cation and/or anion. Thus, an ionic liquid may be composed of more than one species of cation and one species of anion. An ionic liquid may further be composed of one species of cation, and more than one species of anion. Thus, the mixed salts used in the present invention can comprise mixed salts containing anions and cations.

The term “ionic liquid” includes both compounds having high melting temperature and compounds having low melting points, e.g. at or below room temperature (i.e. 15 to 30° C.). The latter are often referred to as “room temperature ionic liquids” and are often derived from organic salts having pyridinium- and imidazolium-based cations. A feature of ionic liquids is that they have particularly low (essentially zero) vapour pressures. Many organic ionic liquids have low melting points, for example, less than 100° C., particularly less than 80° C., and around room temperature, e.g. 15 to 30° C., and some have melting points well below 0° C. For the purposes of the present invention, it is desirable that the organic ionic liquid has a melting point of 250° C. or less, preferably 150° C. or less, more preferably 100° C. and even more preferably 80° C. or less, although any compound that meets the criteria of being a salt consisting of an anion and cation, and has antimicrobial properties, may be used in the compositions of the present invention.

Ionic liquids are most widely known as solvents because their non-volatility, low flammability, applicability at wide temperature ranges and the possibility of recycling make them environmentally friendly. Such solvents are greatly desired for industrial processes.

Although not wishing to be bound by any theory, the mechanism of antimicrobial action demonstrated by the ionic liquids of the present invention is thought to be due to the disruption of intermolecular interactions by hydrocarbyl chains such as alkyl-like moieties (preferably alkyl chains) present in the ionic liquid. In bacteria, in particular, this can cause the dissociation of cellular membrane bilayers, thereby inducing leakage of cellular contents, as well as the dissociation of other biomolecular structures within the bacterial cell. Enzymes within bacterial cells may also be denatured by conformational changes that result from interactions with ionic liquids, thereby disrupting cellular respiratory and metabolic processes. If the energy source of the cell is disrupted, the cell cannot maintain osmotic pressure, and the microbe will quickly die.

It has also been found that the antimicrobial system of the present invention has surprising antimicrobial activity towards biofilms.

Biofilms are complex aggregations of microorganisms growing on a solid surface which are held together by an extracellular matrix of secreted polymeric compounds. Biofilms may be formed of a single microbial species, but more often biofilms are a complex aggregation of bacteria, fungi, algae, protozoa, and other debris.

The polymeric matrix of a biofilm protects the cells within it and, as a consequence, microbes within a biofilm often have very different properties from free-floating (planktonic) bacteria as the dense extracellular matrix and an outer layer of microbial cells protects microbes in the interior of the film. Furthermore, it has been found that different genes are activated in bacteria within biofilms, which makes bacteria within biofilms phenotypically different organisms to the corresponding planktonic bacteria. The US National Institutes for Health (NIH) have estimated that up to 80% of all chronic human infections are biofilm-mediated and that 99.9% of bacteria in aquatic ecosystems live as biofilm communities.

One important effect of the biofilm environment is to provide microbes with increased resistance to detergents and antibiotics, and in some cases resistance can be increased as much as 1000 fold compared to the corresponding planktonic bacteria. It is therefore difficult to extrapolate planktonic bactericidal data to environmental or clinical scenarios where the majority of bacterial growth, for example on substrate surfaces, is in the form of biofilms. As the microbes within the biofilm remain healthy, the film is able to regrow, and repeated use of antimicrobial agents on biofilms may cause microbes within the film to develop an increased resistance to biocides. Thus, conventional disinfecting measures are often ineffective to deal with biofilm contamination. Further, in many cases, the high doses of biocide that are required to remove biofilm contamination are damaging to the environment and hazardous to human and animal health.

Biofilms are common in nature, and biofilms on surfaces (such as floors, food preparation surfaces and sanitaryware) are a significant source of microbial infections. This is of particular concern in industrial food preparation premises, and in hospitals, where patients often already have decreased resistance to pathogens. In addition, biofilms can form in the interior of pipes in plumbing systems and industrial machinery leading to clogging, product contamination, equipment failure, and productivity losses from equipment downtime for cleaning or replacement of fouled parts.

The antimicrobial system of the present invention therefore provides a useful and highly effective alternative to conventional methods for eradicating biofilm contamination. In particular, the present invention provides a number of benefits over known methods of biofilm eradication. In particular, the antimicrobial system of the invention provides increased biocidal activity towards biofilms, and prolonged activity to prevent recontamination. As noted above, bacteria within biofilms are known to demonstrate as much as 1000 fold increase in resistance to conventional disinfectants. By contrast, the antimicrobial system of the present invention has been found in some cases to have a minimum biofilm eradication concentration (MBEC) which is as low as the minimum bactericidal concentration (MBC) for the corresponding bacteria in the planktonic state.

In addition to their broad-spectrum antimicrobial activity, ionic liquids have a number of physical and chemical properties that make them particularly suitable for use as disinfectants. In particular, ionic liquids have negligible vapour pressure, and therefore their disinfecting capacity is not depleted through evaporation. Accordingly, antimicrobial activity is maintained over extended periods. Additionally, the lack of vapour pressure of ionic liquids means that ionic liquid disinfectants have no unpleasant or harmful odours. Unlike oxidising disinfectants (such as bleach), ionic liquid disinfectants do not harm surfaces, are generally not inactivated by sunlight and do not significantly degrade over time.

However, such ionic liquids tend to be highly viscous which makes them difficult to spread over a substrate surface, and even more difficult to apply as a thin layer. The inventors of the present invention, in addition to identifying ionic liquids having superior antimicrobial properties have also developed compositions which allow the ionic liquids to be applied as thin, preferably uniform, layers which are suitable for industrial use such as in hospitals, and domestic use such as private homes.

According to an aspect of the present invention there is provided the use of an ionic liquid having the formula:

[Cat⁺][X⁻]
wherein: [Cat⁺] is one or more cationic species comprising at least one quinolinium or isoquinolinium cationic species; and
   [X⁻] is one or more anionic species

as an antibacterial agent.

In accordance with the present invention, the at least one quinolinium or isoquinolinium cationic species may be selected from:

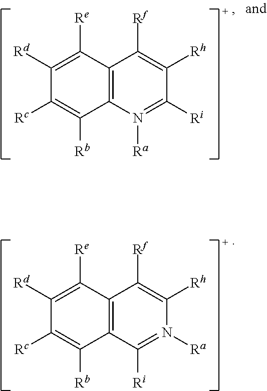

wherein: R^{a}, R^{b}, R^{c}, R^{d}, R^{e}, R^{f}, R^{h} and Rⁱ can be the same or different, and are each independently selected from hydrogen, a C₁ to C₄₀ linear or branched alkyl group, a C₃ to C₈ cycloalkyl group, or a C₆ to C₁₀ aryl group, or any two of R^{b}, R^{c}, R^{d}, R^{e}, R^{f}, R^{h} and Rⁱ attached to adjacent carbon atoms form a methylene chain —(CH₂)_{q}— wherein q is from 8 to 20, wherein said alkyl, cycloalkyl or aryl groups, or said methylene chain, are unsubstituted or may be substituted by one to three groups selected from: C₁ to C₆ alkoxy, C₆ to C₁₀ aryl, CN, OH, NO₂, C₇ to C₃₀ aralkyl and C₇ to C₃₀ alkaryl.

Preferably, R^{a} is selected from C₁ to C₃₀ linear or branched alkyl, or hydrogen. More preferably, R^{a} is selected from C₁ to C₃₀ linear or branched alkyl, more preferably C₂ to C₂₀ linear or branched alkyl, still more preferably C₄ to C₁₈ linear or branched alkyl, still more preferably C₈ to C₁₈ linear or branched alkyl, and most preferably C₈ to C₁₄ linear or branched alkyl.

Further examples include wherein R^{a} is selected from ethyl, butyl, hexyl, octyl, nonyl, decyl, undecyl, dodecyl, tridecyl, tetradecyl, pentadecyl, hexadecyl, heptadecyl and octadecyl.

In a further preferred embodiment, R^{a} is selected from C₁ to C₃₀ linear or branched alkyl and C₁ to C₁₅ alkoxyalkyl.

In a further embodiment, R^{b}, R^{c}, R^{d}, R^{e}, R^{f}, R^{h} and Rⁱ are each independently selected from hydrogen or a C₁ to C₃₀ linear or branched alkyl group. More preferably R^{b}, R^{c}, R^{d}, R^{e}, R^{f}, R^{h} and Rⁱ are each independently selected from hydrogen or a C₁ to C₁₂ linear or branched alkyl group, more preferably from hydrogen or a C₁ to C₆ linear or branched alkyl group, and still more preferably hydrogen or a methyl group.

More preferably, one of R^{b}, R^{c}, R^{d}, R^{e}, R^{f}, R^{h} and Rⁱ is selected from hydrogen or a C₁ to C₃₀ linear or branched alkyl group, and the remainder of R^{b}, R^{c}, R^{d}, R^{e}, R^{f}, R^{h} and Rⁱ are each hydrogen. Preferably, one of R^{b}, R^{c}, R^{d}, R^{e}, R^{f}, R^{h} and Rⁱ is selected from hydrogen or a C₁ to C₁₂ linear or branched alkyl group, still more preferably hydrogen or a C₁ to C₆ linear or branched alkyl group, and most preferably hydrogen or a methyl group, and the remainder of R^{b}, R^{c}, R^{d}, R^{e}, R^{f}, R^{h} and Rⁱ are each hydrogen.

Preferably, R^{d} is selected from hydrogen or a C₁ to C₃₀ linear or branched alkyl group, more preferably hydrogen or a C₁ to C₁₂ linear or branched alkyl group, still more preferably hydrogen or a C₁ to C₆ linear or branched alkyl group, and most preferably hydrogen or a methyl group.

Still more preferably, R^{d} is selected from hydrogen or a C₁ to C₃₀ linear or branched alkyl group and R^{b}, R^{c}, R^{e}, R^{f}, R^{h} and Rⁱ are each hydrogen. Preferably, R^{d} is selected from hydrogen or a C₁ to C₁₂ linear or branched alkyl group, still more preferably hydrogen or a C₁ to C₆ linear or branched alkyl group, and most preferably hydrogen or a methyl group, and R^{b}, R^{c}, R^{e}, R^{f}, R^{h} and Rⁱ are each hydrogen.

Examples of preferred quinolinium and isoquinolinium cations which may be used in accordance with this aspect of the present invention include: N—(C₈-C-₁₈)alkyl-quinolinium, N—(C₈-C-₁₈)alkyl-isoquinolinium, N—(C₈-C-₁₈)alkyl-6-methylquinolinium and N—(C₈-C₁₈)alkyl-6-methylisoquinolinium cations; more preferably N—(C₁₂-C-₁₆)alkyl-quinolinium, N—(C₁₂-C₁₆)alkyl-isoquinolinium, N—(C₁₂-C-₁₆)alkyl-6-methylquinolinium and N—(C₁₂-C₁₆)alkyl-6-methylisoquinolinium cations.

Further examples include N-octylquinolinium, N-decylquinolinium, N-dodecylquinolinium, N-tetradecylquinolinium, N-octylisoquinolinium, N-decylisoquinolinium, N-dodecyl-isoquinolinium, N-tetradecylisoquinolinium, N-octyl-6-methylquinolinium, N-decyl-6-methylquinolinium, N-dodecyl-6-methyl-quinolinium, N-tetradecyl-6-methylquinolinium, N-octyl-6-methylisoquinolinium, N-decyl-6-methylisoquinolinium, N-dodecyl-6-methylisoquinolinium, and N-tetradecyl-6-methylisoquinolinium.

N-tetradecylisoquinolinium and N-tetradecyl-6-methylquinolinium cations are particularly preferred.

In accordance with the present invention, [Cat⁺] may represent a single quinolinium or isoquinolinium cationic species as described above.

Alternatively, [Cat⁺] may represent a combination of two or more quinolinium or isoquinolinium cationic species as described above.

In a further embodiment, [Cat⁺] may comprise one or more quinolinium or isoquinolinium cationic species as described above, together with one or more further cationic species selected from: imidazolium, pyridinium, pyrazolium, thiazolium, isothiazolium, azathiazolium, oxathiazolium, oxazinium, oxazolium, oxaborolium, dithiazolium, triazolium, selenazolium, oxaphospholium, pyrrolium, borolium, furanium, thiophenium, phospholium, pentazolium, indolium, indolinium, iso-oxazolium, iso-triazolium, tetrazolium, benzofuranium, thiadiazolium, pyrimidinium, pyrazinium, pyridazinium, piperazinium, piperidinium, morpholinium, pyranium, annulenium, phthalazinium, quinazolinium, quinoxalinium, thiazinium, azaannulenium, ammonium, pyrrolidinium, diazabicycloundecenium, diazabicyclononenium, diazabicyclodecenium, phosphonium or triazadecenium.

In accordance with this embodiment of the invention, the one or more further cationic species may be selected from:

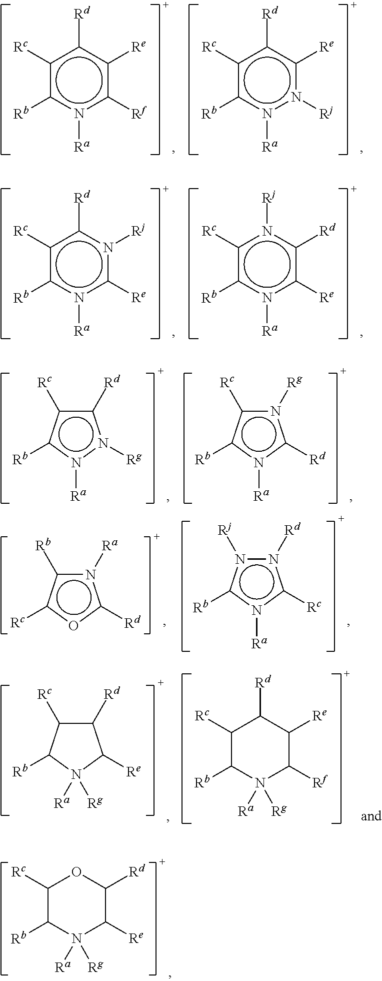

wherein: R^{a}, R^{b}, R^{c}, R^{d}, R^{e}, R^{f}, R^{g}, R^{h}, Rⁱ and R^{j} can be the same or different, and are each independently selected from hydrogen, a C₁ to C₄₀, straight chain or branched alkyl group, a C₃ to C₈ cycloalkyl group, or a C₆ to C₁₀ aryl group, wherein said alkyl, cycloalkyl or aryl groups are unsubstituted or may be substituted by one to three groups selected from: C₁ to C₆ alkoxy, C₆ to C₁₀ aryl, CN, OH, NO₂, C₇ to C₃₀ aralkyl and C₇ to C₃₀ alkaryl, or any two of R^{b}, R^{c}, R^{d}, R^{e}, R^{f}, R^{h} and Rⁱ attached to adjacent carbon atoms form a methylene chain —(CH₂)_{q}— wherein q is from 8 to 20, and wherein R^{j} may be absent.

More preferably, the one or more further cationic species may be selected from:

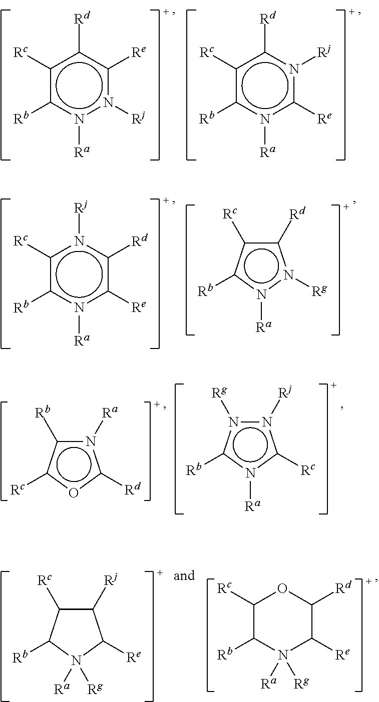

wherein: R^{a}, R^{b}, R^{c}, R^{d}, R^{e}, R^{f}, R^{g}, R^{h}, Rⁱ and R^{j} are as defined above.

Still more preferably, the one or more further cationic species may be selected from:

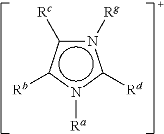

wherein: R^{a}, R^{b}, R^{c}, R^{d}, R^{g} are as defined above.

Where present, R^{a}, R^{g} and R^{j} are preferably independently selected from C₁ to C₃₀ linear or branched alkyl, and one of R^{a}, R^{g} and R^{j} may also be hydrogen.

R^{a} is preferably selected from C₂ to C₂₀ linear or branched alkyl, more preferably C₄ to C₁₈ linear or branched alkyl, still more preferably C₈ to C₁₈ linear or branched alkyl, and most preferably C₈ to C₁₄ linear or branched alkyl.

Where present R^{g} and R^{j} are preferably independently selected from C₁ to C₁₀ linear or branched alkyl, more preferably C₁ to C₅ linear or branched alkyl, and most preferably a methyl group.

Further examples include wherein one of R^{a}, R^{g} and R^{j} is selected from ethyl, butyl, hexyl, octyl, nonyl, decyl, undecyl, dodecyl, tridecyl, tetradecyl, pentadecyl, hexadecyl, heptadecyl and octadecyl.

In a further preferred embodiment, R^{a}, R^{g} and R^{j} may each be independently selected, where present, from C₁ to C₃₀ linear or branched alkyl, and C₁ to C₁₅ alkoxyalkyl.

Where R^{a} and R^{g} are both present, they are each preferably independently selected from C₁ to C₃₀ linear or branched alkyl, and one of R^{a} and R^{g} may also be hydrogen. More preferably, one of R^{a} and R^{g} may be selected from C₂ to C₂₀ linear or branched alkyl, more preferably C₄ to C₁₈ linear or branched alkyl, still more preferably C₈ to C₁₈ linear or branched alkyl, and most preferably C₈ to C₁₄ linear or branched alkyl, and the other one of R^{a} and R^{g} may be selected from C₁ to C₁₀ linear or branched alkyl, more preferably C₁ to C₅ linear or branched alkyl, and most preferably a methyl group.

In accordance with this aspect of the invention, the ionic liquid may comprise one or more anion species selected from: [BE]⁻, [PF₆]⁻, [SbF₆]⁻, [F]⁻, [Cl]⁻, [Br]⁻, [NO₃]⁻, [NO₂]⁻, [H₂PO₄]⁻, [HPO₄]²⁻, [R^{x}₂PO₄]⁻, [R^{x}PO₄]²⁻, [R^{x}₃PF₃]⁻, [R^{x}₂P(O)O]⁻ [HSO₃]⁻, [HSO₄]⁻, [R^{x}SO₃]⁻, [R^{x}SO₄]⁻, [SO₄]²⁻, [H₃CO(CH₂)₂O(CH₂)OSO₃]⁻, [BBDB]⁻, [BOB]⁻, [(CF₃SO₂)₃C]⁻, [Co(CO₄)]⁻, [(CN)₂N]⁻, [(CF₃)₂N]⁻, [(R^{x}SO₂)₂N]⁻, [SCN]⁻, [H₃C(OCH₂CH₂)ₙOSO₃]⁻, [R^{x}O₂CCH₂CH(CO₂R^{x})SO₃]⁻, [R^{x}CO₂]⁻, lactate, and docusate; wherein each R^{x} is independently selected from (C₁-C₂₀)alkyl, (C₆-C₁₀)aryl, and (C₁-C₁₀)alkyl(C₆-C₁₀)aryl; and further wherein said alkyl, aryl and alkylaryl groups may comprise one or more substituents independently selected from F, Cl and I.

In a preferred embodiment, the ionic liquid may comprise one or more anion species selected from: [Cl]⁻, [Br]⁻, [I]⁻, [H₂PO₄]⁻, [HPO₄]²⁻, [R^{x}₂PO₄]⁻, [R^{x}₂P(O)O⁻], [R^{x}SO₃]⁻, [CH₃SO₃]⁻, [R^{x}SO₄]⁻, [CH₃SO₄]⁻, [C₂H₅SO₄]⁻, [SO₄]²⁻, [R^{x}O₂CCH₂CH(CO₂R^{x})SO₃]⁻, [R^{x}CO₂]⁻, [C₆H₄CO₂]⁻, lactate and docusate; wherein R^{x} is as defined above. More preferably, the ionic liquid may comprise one or more anion species selected from: [Cl]⁻, [Br]⁻, and [I]⁻.

In a further preferred embodiment, the ionic liquid may comprise one or more anion species selected from: [BF₄]⁻, [PF₆]⁻, [BBDB]⁻, [BOB]⁻, [N(CF₃)₂]⁻, [(CF₃SO₂)₂N]⁻, [(CF₃SO₂)₃C]⁻, [(C₂F₅)₃PF₃]⁻, [(C₃F₇)₃PF₃]⁻, [(C₂F₅)₂P(O)O]⁻, [SbF₆]—, [Co(CO)₄]⁻, [NO₃]⁻, [NO₂]⁻, [CF₃SO₃]⁻, [CH₃SO₃]⁻, [C₈H-₁₇OSO₃]⁻, and tosylate.

It will be appreciated that the present invention is not limited to ionic liquids comprising anions and cations having only a single charge. Thus, the formula [Cat⁺][X⁻] is intended to encompass ionic liquids comprising, for example, doubly, triply and quadruply charged anions and/or cations. The relative stoichiometric amounts of [Cat⁺] and [X⁻] in the ionic liquid are therefore not fixed, but can be varied to take account of cations and anions with multiple charges. For example, the formula [Cat⁺][X⁻] should be understood to include ionic liquid species having the formulae [Cat⁺]₂[X²⁻]; [Cat²⁺][X⁻]₂; [Cat²⁺][X²⁻]; [Cat⁺]₃[X³⁻]; [Cat³⁺][X⁻]₃ and so on.

It will also be appreciated that the present invention is not limited to ionic liquids comprising a single cation and a single anion. Thus, [Cat⁺] may, in certain embodiments, represent two or more cations, such as a mixture of an N-alkylquinolinium cation and a 1-ethyl-3-methylimidazolium cation. Similarly, [X⁻] may, in certain embodiments, represent two or more anions, such as a mixture of chloride ([Cl]⁻) and bistriflimide ([N(SO₂CF₃)₂]⁻) anions.

Where [Cat⁺] represents a mixture of cations, the mixture of cations preferably comprises at least 10 mol % of a quinolinium or isoquinolinium cation as described above, or a mixture thereof, more preferably at least 20 mol %, more preferably at least 30 mol %, more preferably at least 40 mol %, more preferably at least 50 mol %, more preferably at least 60 mol %, more preferably at least 70 mol %, more preferably at least 80 mol %, and still more preferably at least 90 mol %. Most preferably the mixture of cations comprises at least 95 mol % of a quinolinium or isoquinolinium cation as described above, or a mixture thereof.

In a further embodiment, the ionic liquid may comprise a metal ion selected from silver, copper, tin and zinc ions. Preferably, the metal ion is selected from Ag⁺, Cu⁺, Cu²⁺, Sn²⁺ and Zn²⁺ ions, still more preferably the metal ion is selected from Ag⁺, Cu⁺ and Cu²⁺ ions, and most preferably the metal ion is selected from Ag⁺ and Cu²⁺ ions.

It will be appreciated that ionic liquids comprising metal ions according to the present invention necessarily comprise a sufficient amount of the anionic species [X⁻] so as to maintain charge balance.

It will be appreciated by those of skill in the art that metal ions in ionic liquids may take the form of complexes, where the term “complex” is intended to refer to a metal ion surrounded by one or more ligands. In preferred embodiments, the ligands are the same as one of the ionic liquid anions. In further preferred embodiments the metal ion is in the form of a metal halide complex, more preferably a metal chloride or bromide complex.

The present invention further relates to the use of an ionic liquid comprising a species having the formula:

[Cat⁺][M⁺][X⁻]
wherein: [Cat⁺] is one or more cationic species comprising at least one quinolinium or isoquinolinium cationic species, as described above;
   [M⁺] is one or more metal ions selected from silver, copper, tin and zinc ions; and
[X⁻] is one or more anionic species, as described above;

as an antibacterial agent.

As above, the relative stoichiometric amounts of each of [Cat⁺], [M⁺], and [X⁻] in the species [Cat⁺][M⁺][X⁻], are not limited, and may be determined by the skilled person taking into account the charge on each of [Cat⁺], [M⁺], and [X⁻] (each of which may have a single or multiple charge), and charge balance considerations, as well as the coordination number of the metal ion.

Most preferably the ionic liquid comprises a species having the formula [Cat⁺][Ag⁺][Br⁻]₂, the formula [Cat⁺]₂[Cu²⁺][Cl⁻]₄, or the formula [Cat⁺][Cu⁺][Cl⁻]₂. More preferably, the ionic liquid comprises a species having the formula [Cat⁺][Ag⁺][Br⁻]₂ or the formula [Cat⁺]₂[Cu²⁺][Cl⁻]₄. Thus, the metal complex preferably has the formula [Ag⁺][Br⁻]₂ or the formula [Cu²⁺][Cl⁻]₄.

In a further aspect, the present invention provides the use of an ionic liquid having the formula [Cat⁺][X⁻]
wherein [Cat⁺] is one or more heterocyclic cationic species selected from: imidazolium, pyridinium, pyrazolium, thiazolium, isothiazolium, azathiazolium, oxathiazolium, oxazinium, oxazolium, oxaborolium, dithiazolium, triazolium, selenazolium, oxaphospholium, pyrrolium, borolium, furanium, thiophenium, phospholium, pentazolium, indolium, indolinium, iso-oxazolium, iso-triazolium, tetrazolium, benzofuranium, thiadiazolium, pyrimidinium, pyrazinium, pyridazinium, piperazinium, piperidinium, morpholinium, pyranium, annulenium, phthalazinium, quinazolinium, quinoxalinium, thiazinium, azaannulenium, pyrrolidinium, diazabicycloundecenium, diazabicyclononenium, diazabicyclodecenium and triazadecenium; and
   [X⁻] is one or more anionic species,

and further comprising a metal ion selected from silver, copper, tin and zinc ions, as an antibacterial agent.

Preferably, the metal ion is selected from Ag⁺, Cu⁺, Cu²⁺, Sn²⁺ and Zn²⁺ ions, still more preferably the metal ion is selected from Ag⁺, Cu⁺ and Cu²⁺ ions, and most preferably the metal ion is selected from Ag⁺ and Cu²⁺ ions.

As above, the metal ions may take the form of complexes. In preferred embodiments, the ligands are the same as one of the ionic liquid anions.

In accordance with this aspect of the invention, [Cat⁺] is preferably one or more cationic species selected from:

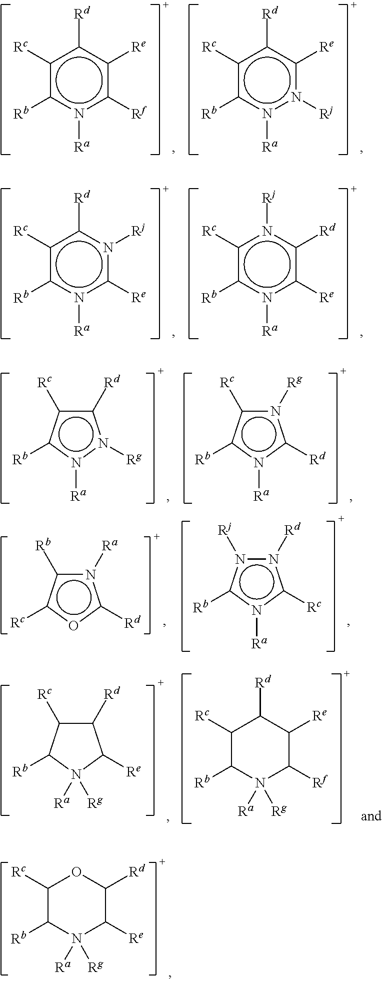

wherein: R^{a}, R^{b}, R^{c}, R^{d}, R^{e}, R^{f}, R^{g}, R^{h}, Rⁱ and R^{j} can be the same or different, and are each independently selected from hydrogen, a C₁ to C₄₀, straight chain or branched alkyl group, a C₃ to C₈ cycloalkyl group, or a C₆ to C₁₀ aryl group, wherein said alkyl, cycloalkyl or aryl groups are unsubstituted or may be substituted by one to three groups selected from: C₁ to C₆ alkoxy, C₆ to C₁₀ aryl, CN, OH, NO₂, C₇ to C₃₀ aralkyl and C₇ to C₃₀ alkaryl, or any two of R^{b}, R^{c}, R^{d}, R^{e}, R^{f}, R^{h} and Rⁱ attached to adjacent carbon atoms form a methylene chain —(CH₂)_{q}— wherein q is from 8 to 20, and wherein R^{j} may be absent.

More preferably, [Cat⁺] is one or more cationic species selected from:

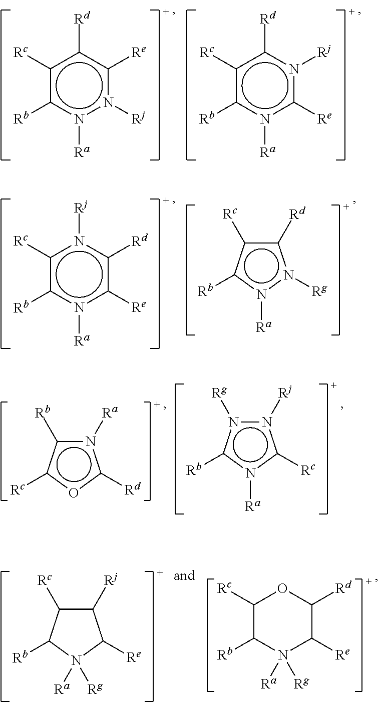

wherein: R^{a}, R^{b}, R^{c}, R^{d}, R^{e}, R^{f}, R^{g}, R^{h}, Rⁱ and R^{j} are as defined above.

In another preferred embodiment, [Cat⁺] is selected from:

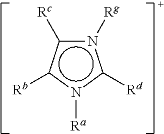

wherein: R^{a}, R^{b}, R^{c}, R^{d}, R^{g} are as defined above.

Where present, R^{a}, R^{g} and R^{j} are preferably independently selected from C₁ to C₃₀ linear or branched alkyl, and one of R^{a}, R^{g} and R^{j} may also be hydrogen.

R^{a} is preferably selected from C₂ to C₂₀ linear or branched alkyl, more preferably C₄ to C₁₈ linear or branched alkyl, still more preferably C₈ to C₁₈ linear or branched alkyl, and most preferably C₈ to C₁₄ linear or branched alkyl.

Where present R^{g} and R^{j} are preferably selected from C₁ to C₁₀ linear or branched alkyl, more preferably C₁ to C₅ linear or branched alkyl, and most preferably a methyl group.

Further examples include wherein one of R^{a}, R^{g} and R^{j} is selected from ethyl, butyl, hexyl, octyl, nonyl, decyl, undecyl, dodecyl, tridecyl, tetradecyl, pentadecyl, hexadecyl, heptadecyl and octadecyl.

In a further preferred embodiment, R^{a}, R^{g} and R^{j} may each be independently selected, where present, from C₁ to C₃₀ linear or branched alkyl, and C₁ to C₁₅ alkoxyalkyl.

Where R^{a} and R^{g} are both present, they are each preferably independently selected from C₁ to C₃₀ linear or branched alkyl, and one of R^{a} and R^{g} may also be hydrogen. More preferably, one of R^{a} and R^{g} may be selected from C₂ to C₂₀ linear or branched alkyl, more preferably C₄ to C₁₈ linear or branched alkyl, still more preferably C₈ to C₁₈ linear or branched alkyl, and most preferably C₈ to C₁₄ linear or branched alkyl, and the other one of R^{a} and R^{g} may be selected from C₁ to C₁₀ linear or branched alkyl, more preferably C₁ to C₅ linear or branched alkyl, and most preferably a methyl group.

In accordance with this aspect of the present invention, [X⁻] may be selected from: [BF₄]⁻, [PF₆]⁻, [SbF₆]⁻, [F]⁻, [Cl]⁻, [Br]⁻, [I]⁻, [NO₃]⁻, [NO₂]⁻, [H₂PO₄]⁻, [HPO₄]²⁻, [R^{x}₂PO₄]⁻, [R^{x}PO₄]²⁻, [R^{x}₃PF₃]⁻, [R^{x}₂P(O)O]⁻, [HSO₃]⁻, [HSO₄]⁻, [R^{x}SO₃]⁻, [R^{x}SO₄]⁻, [SO₄]²⁻, [H₃CO(CH₂)₂O(CH₂)OSO₃]⁻, [bis(1,2-benzenedioxy)borate]⁻ ([BBDB]⁻), [bis(oxalato)-borate]⁻ ([BOB]⁻), [(CF₃SO₂)₃C]⁻, [Co(CO₄)]⁻, [(CN)₂N]⁻, [(CF₃)₂N]⁻, [(R^{x}SO₂)₂N]⁻, [SCN]⁻, [H₃C(OCH₂CH₂)ₙOSO₃]⁻, [R^{x}O₂CCH₂CH(CO₂R^{x})SO₃]⁻, [R^{x}CO₂]⁻, lactate, and docusate; wherein each R^{x} is independently selected from (C₁-C₂₀)alkyl, (C₆-C₁₀)aryl, and (C₁-C₁₀)alkyl(C₆-C₁₀)aryl; and further wherein said alkyl, aryl and alkylaryl groups may comprise one or more substituents independently selected from F, Cl and I.

Preferably, [X⁻] may be selected from [Cl]⁻, [Br]⁻, [I]⁻, [H₂PO₄]⁻, [HPO₄]²⁻, [R^{x}₂PO₄]⁻, [R^{x}₂P(O)O⁻], [R^{x}SO₃]⁻, [CH₃SO₃]⁻, [R^{x}SO₄]⁻, [CH₃SO₄]⁻, [C₂H₅SO₄]⁻, [SO₄]²⁻, [R^{x}O₂CCH₂CH(CO₂R^{x})SO₃]⁻, [R^{x}CO₂]⁻, benzoate, substituted benzoate, lactate and docusate; wherein R^{x} is as defined above. Most preferably, [X⁻] is selected from [Cl]⁻, [Br]⁻ and [I]⁻.

[X⁻] may also preferably be selected from [BF₄]⁻, [PF₆]⁻, [BBDB]⁻, [BOB]⁻, [N(CF₃)₂]⁻, [(CF₃SO₂)₂N]⁻, [(CF₃SO₂)₃C]⁻, [(C₂F₅)₃PF₃]⁻, [(C₃F₇)₃PF₃]⁻, [(C₂F₅)₂P(O)O]⁻, [SbF₆]—, [Co(CO)₄]⁻, [NO₃]⁻, [NO₂]⁻, [CF₃SO₃]⁻, [CH₃SO₃]⁻, [C₈H₁₇OSO₃]⁻, and tosylate.

Examples of preferred ionic liquids according to this aspect of the present invention have the formula C_{y}MIMX, wherein C_{y}MIM denotes a 1-alkyl-3-methylimidazolium cation wherein the alkyl group is a straight chain alkyl group having y carbon atoms; y is 4 to 18, preferably 8 to 14; and X is a halide anion which is most preferably chloride.

This aspect of the invention is not limited to ionic liquids comprising anions and cations having only a single charge. Thus, the formula [Cat⁺][X⁻] is intended to encompass ionic liquids comprising, for example, doubly, triply and quadruply charged anions and/or cations. The relative stoichiometric amounts of [Cat⁺] and [X⁻] in the ionic liquid are therefore not fixed, but can be varied to take account of cations and anions with multiple charges. For example, the formula [Cat⁺][X⁻] should be understood to include ionic liquids having the formulae [Cat⁺]₂[X²⁻], [Cat²⁺][X⁻]₂, [Cat²⁺][X²⁻], [Cat⁺]₃[X³⁻], [Cat³⁺][X⁻]₃ and so on.

This aspect of the present invention is also not limited to ionic liquids comprising a single cation and a single anion. Thus, [Cat⁺] may, in certain embodiments, represent two or more cations, such as a statistical mixture of 1,3-dimethylimidazolium, 1-ethyl-3-methylimidazolium and 1,3-diethylimidazolium cations. Similarly, [X⁻] may, in certain embodiments, represent two or more anions, such as a mixture of chloride ([Cl]⁻) and bistriflimide ([N(SO₂CF₃)₂]⁻) anions.

The present invention further relates to the use of ionic liquids comprising a species having the formula:

[Cat⁺][M⁺][X⁻]
wherein: [Cat⁺] is one or more heterocyclic cationic species, as defined above;
   [M⁺] is one or more metal ions selected from silver, copper, tin and zinc ions; and
   [X⁻] is one or more anionic species, as described above,

as antibacterial agents.

Preferred silver and copper containing ionic liquids according to this aspect of the present invention are those comprising a species having the formulae C₍₈₋₁₈₎MIMAgBr₂ and (C₍₈₋₁₈₎MIM)₂CuCl₄. Even more preferred are those comprising a species having the formulae C₍₁₂₋₁₆₎MIMAgBr₂ and (C₍₁₂₋₁₆₎MIM)₂CuCl₄.

The relative stoichiometric amounts of each of [Cat⁺], [M⁺], and [X⁻] in the species [Cat⁺][M⁺][X⁻], are not limited, and may be determined by the skilled person taking into account the charge on each of [Cat⁺], [M⁺], and [X⁻] (each of which may have a single or multiple charge), and charge balance considerations. For example, in the case where [Cat⁺] and [M⁺] are both singly charged, [X⁻] may be a doubly charged anion or two singly charged anions. In another example, where [Cat⁺] is singly charged and [M⁺] is doubly charged, [X⁻] may be a triply charged anion, a doubly charged anion and a singly charged anion, or three singly charged anions. The stoichiometry of the species [Cat⁺][M⁺][X⁻] may also be affected by the number of anions that form a complex with the metal ion, i.e. the coordination number of the metal ion.

The ionic liquids described above may be used in the form of antimicrobial systems comprising:

(a) a film-enhancing composition;

(b) a viscosity controlling agent; and

(c) an ionic liquid, as defined above.

The ionic liquid should be present in the antimicrobial systems in an amount sufficient to retard and/or kill microbes. Such amounts may be easily determined by a person of skill in the art using known testing methods, for example ASTM E2180-01, and may be tailored according to the microbes of interest. Suitable minimum inhibitory concentrations (MIC) are generally from 1 μM to 10000 μM, more preferably 10 μM to 1000 μM, still more preferably 20 μM to 500 μM, and most preferably 25 μM to 100 μM.

A preferred ionic liquid according to an aspect of the present invention is C₈MIMCI. MIC values for this ionic liquid are in the range of 1200 to 1800 μM for each of MRSA, P. aeroginosa, K. Aeroginosa, P. mirabilis, B. cenocepacia, and C. tropicalis; 300 to 450 μM for S. Epidermidis; and 600 to 900 μM for E. coli. Minimum bactericidal concentrations (MBC) values are in the range of 1200 to 1700 μM for each of MRSA, S. Epidermidis, E. coli, P. aeroginosa, K. Aeroginosa, P. mirabilis, B. cenocepacia, and C. tropicalis.

Another preferred ionic liquid according to an aspect of the present invention is C₁₀MIMCI. Suitable MIC values for this ionic liquid are in the range of 130 to 190 μM for MRSA; 30 to 50 μM for S. Epidermidis; 260 to 380 μM for E. coli and C. tropicalis; 1000 to 1500 μM for P. aeroginosa, P. mirabilis and B. cenocepacia; and 520 to 760 μM for K. Aeroginosa. MBC values are in the range of 520 to 760 μM for MRSA and S. Epidermidis; 1000 to 1500 μM for each of E. coli, P. aeroginosa, K. Aeroginosa, P. mirabilis and B. cenocepacia; and 260 to 380 μM for C. tropicalis.

A further preferred ionic liquid according to an aspect of the present invention is C₁₂MIMCI. Suitable MIC values for this ionic liquid are in the range of 25 to 45 μM for MRSA and S. Epidermidis; 50 to 90 μM for E. coli, K. Aeroginosa and C. tropicalis; 400 to 720 μM for P. aeroginosa and P. mirabilis; and 200 to 360 μM for B. cenocepacia. Suitable MBC values are in the range of 200 to 360 μM for MRSA; 100 to 180 μM for S. Epidermidis; and K. Aeroginosa; 50 to 90 μM for E. coli and C. tropicalis; 1000 to 1600 μM for P. mirabilis; and 450 to 720 μM for B. cenocepacia.

Another preferred ionic liquid according to an aspect the present invention is C₁₄MIMCI. Suitable MIC values for this ionic liquid are in the range of 25 to 40 μM for MRSA, S. Epidermidis, E. coli, and K. Aeroginosa; 200 to 320 μM for P. aeroginosa and P. mirabilis; 100 to 160 μM for B. cenocepacia; and 50 to 80 μM for C. tropicalis. Suitable MBC values are in the range of 25 to 40 μM for MRSA, S. Epidermidis and E. coli; 200 to 320 μM for P. aeroginosa and B. cenocepacia; 50 to 80 μM for K. Aeroginosa; 400 to 640 μM for P. mirabilis; and 100 to 160 μM for C. tropicalis.

The viscosity controlling agent for the antimicrobial system may comprise one or more solvents and/or thickening agents. In one embodiment of the present invention, the antimicrobial system is prepared by blending the ionic liquid, the film-enhancing composition and any optional components in a solvent, wherein the solvent is capable of dissolving and/or dispersing the ionic liquid, the film-enhancing composition and any optional components. Thickening agents may also be added where the antimicrobial system lacks suitable viscosity to be applied to substrate surfaces in order to form a layer of suitable thickness, for example, where a higher concentration of the antimicrobial ionic liquid on the surface is desirable. It will be appreciated that mixtures of solvents and thickening agents may be used to obtain desired viscosities, and that such mixing and selecting of suitable solvents/thickening agents is well within the knowledge of the person skilled in the art.

Advantageously, the antimicrobial system of the present invention can be formulated to have a variety of viscosities and a wide range of percent solids depending upon the desired application of the system. In certain applications, it may be desired that the system, once applied, provides a coating on a substrate wherein the substrate is substantially non-porous. In others it may be desired that the system be capable of penetrating at least a portion of the surface of a substrate so that disinfecting and antimicrobial effect may be seen below the surface of a substrate. In the former case, a relatively viscous solution (possibly having a relatively high percent solids content) would preferably be formulated, and the latter, a lower viscosity solution (having a lower percent solids) would be more appropriate.

In view of the above, and for embodiments of the invention wherein the antimicrobial system is desirably capable of being applied as a coating (as may be the case when the substrate is non-porous, e.g. glass, plastic or metal), the system may be formulated to have a Brookfield viscosity of from about 5000 cps to about 100,000 cps, as measured at 75° F. and 20 RPM sheer with a 5, 6, 7 spindle. A particularly suitable system for such applications may have a viscosity of at least about 15,000 cps, or at least about 30,000 cps, or even at least about 50,000 cps. Similarly, the solids content of such a system may be about 55.0 wt %, or up to about 75.0 wt %.

On the other hand, for embodiments of the invention wherein it is desired for the system to penetrate at least a portion of the surface of a substrate to which it is applied, as may be particularly desirable in porous substrates permeable to water and thus susceptible to microbial infestation and growth, e.g. wood, a suitable system will have a Brookfield viscosity of from about 1 cps to about 5000 cps. A particularly suitable system for such applications may have a viscosity of less than about 1000 cps, or even less than about 500 cps. The solids content of such a composition may be less than about 30 wt %, less than 20 wt %, or even less than about 10 wt %.

Suitable solvents for use in the antimicrobial system of the present invention may include water and organic solvents such as alcohols (e.g. ethanol, methanol and isopropanol), ketones (e.g. acetone), esters (e.g. methyl acetate and ethyl acetate), ethers (e.g. dimethyl ether, diethyl ether and tetrahydrofuran), hydrocarbons and mixtures thereof. Preferably the solvent is selected from water, methanol, ethanol and mixtures thereof.

Suitable thickening agents include starch, gum arabic, guar gum, and carboxymethylcellulose, including mixtures thereof. A particularly suitable thickening agent is commercially available under the trade designation “NEOCRYL-A1127” from DSM NeoResins, Wilmington, Mass.

Suitable film-enhancing components include one or more of wetting agents, dispersing agents, surfactants, pigments, defoaming agents, coalescing agents, fillers, reinforcing agents, adhesion promoters, plasticisers, flow control agents, antioxidants, UV stabilisers, dyes, and polymers.

Suitable surfactants include “SURFONIC L” series surfactants commercially available from Huntsman Corporation, Salt Lake City, Utah; and the trade designated “ZONYL” surfactants commercially available from E.I. du Pont de Nemours and Company.

Preferably the film-enhancing composition comprises a polymer of effective molecular weight to form a film when applied to a substrate surface. The polymer film may be water-insoluble or water-soluble. Preferably the film is resistant to mild abrasion and to leaching of the ionic liquid when in contact with water or other solvents.

In one embodiment, the polymer is hydrophobic and/or water insoluble. More preferably, the polymer is substantially non-ionic, cationic or anionic.

Suitable hydrophobic, water insoluble film-forming polymers that are also non-ionic or cationic and suitable for use in the film-enhancing compositions of the present invention include: styrene acrylic copolymers, such as those commercially available under the trade names Acronol S702 (BASF, Aktiengesellschaft, Mount Olive, N.J.), PD-330 (H.B. Fuller Company, St. Paul, Minn.), and Res 1018, 1019 and 4040 (Rohm & Hass Company, Philadelphia Pa.); acrylic homopolymers such as commercially available under the trade names Ucar 376 and 351 (Dow Chemical Midland, Mich.) and Res 3077 (Rohm & Haas); styrene butadiene block copolymers, such as those commercially available under the trade name DL313NA (Dow Chemical); ethylene vinyl acetate copolymers, such as those commercially available under the trade names Airflex 400/A405/460 (Air Products and Chemicals, Inc., Allentown, Pa.) and Elvace 1875 (Reichhold Inc., Durham, N.C.); polyvinyl acetate homopolymers, such as those commercially available under the trade names PD-316 (H.B. Fuller Company) and Airflex XX220/230 (Air Products and Chemicals, Inc.); acrylate-acrylonitrile copolymers, such as those commercially available under the trade name Synthemul (various grades, Reichhold Inc.); vinyl acetate-vinyl chloride ethylene copolymers, such as those commercially available under the trade name Airflex 728 (Air Products and Chemicals, Inc.); ethylene vinyl acetate butyl acrylate terpolymers, such as those commercially available under the trade names Airflex 809 and Airflex 811 (Air Products and Chemicals, Inc.); butadiene-acrylonitrile copolymers, such as those commercially available under the trade name Tylac, various grades (Reichhold Inc.); vinyl acrylic-vinyl chloride copolymers, such as those commercially available under the trade name Haloflex 563 (Zeneca Resins, Wilmington, Mass.); polychloroprene polymers and copolymers, such as those commercially available under the trade name DuPont Neoprene latex 115 (E.I. du Pont de Nemours and Company, Wilmington, Del.); and mixtures thereof.

Suitable hydrophobic, water-insoluble film-forming polymers that are anionic and suitable for use in the film-enhancing compositions of the present invention include: styrene acrylic copolymers, such as those commercially available under the trade name PD-600 (BASF); acrylic homopolymers, such as those commercially available under the trade names PD-431, PD-449, PD-483 and PD-2049F (H.B. Fuller Company); vinyl acrylic copolymers, such as those commercially available under the trade names PD-119 and PD-124 (H.B. Fuller Company); styrene butadiene block copolymers such as those commercially available under the trade names NM-565 and ND-422 (BASF) and Rovene 6105 (Mallard Creek Polymers Inc., Charlotte, N.C.); vinylidene chloride-acrylic-vinyl-chloride copolymers, such as those commercially available under the trade names Vycar 660x1 4 and Vycar 460x46 (Noveon Inc., Cleveland, Ohio); water-borne urethane polymers such as NeoRez R-962, 967 and 972 (Zeneca Resins); and mixtures thereof.

In a further embodiment of the invention, the polymer is water-soluble. Such compositions may be useful in environments where the antimicrobial film is expected to remain dry. Suitable water soluble polymers for use in the present invention include polyvinyl alcohols, such as those commercially available from J.T. Baker, Phillipsburg, N.J. and Sigma-Aldrich Company, St. Louis, Mo.; polyvinylpyrrolidinones such as those commercially available from J. T. Baker and those available under the trade names PVP-Kxx from Peakchem, ZheJiang, China (where “xx” indicates the average molecular weight (in 1000s of Daltons) of the polymer—e.g. PVP-K90 and PVP-K30); polyethylene oxides such as those available under the tradenames POLYOX from Dow Chemical Co., Midland, Mich.; sulfonated polyurethanes, and copolymers and mixtures thereof.

The film-enhancing components (especially polymers) may preferably be present in the antimicrobial systems of the present invention in an amount of 1 wt % to 90 wt %, more preferably 10 wt % to 80 wt %, and still more preferably 15 wt % to 75 wt %.

The antimicrobial system may further include an optical reporter, e.g., a fluorophore or an optical brightening agent that enables detection of the composition on a surface by suitable detection devices such as irradiation by an ultraviolet or visible light source.

According to a further aspect of the present invention, there is provided a method of disinfecting a substrate which comprises applying an ionic liquid or an antimicrobial system as defined above to a substrate and allowing the ionic liquid or the antimicrobial system to remain in contact with the substrate for a period of time. In one embodiment, the ionic liquid or the antimicrobial system is applied to the surface of a substrate. In a further embodiment, the ionic liquid or the antimicrobial system is applied to a substrate such that it permeates pores within the substrate.

The present invention further provides a method of disinfecting a substrate (including the surface and/or pores) which comprises applying an ionic liquid or an antimicrobial system as defined above to the surface and allowing the ionic liquid or the antimicrobial system to remain in contact with said surface for a period of time. The ionic liquid or the antimicrobial system may be applied to the substrate by any suitable method, such as spraying, brushing, painting, rolling, or wiping the antimicrobial system onto the substrate, or by immersion of a substrate to be disinfected in a bath of the ionic liquid or the antimicrobial system.

Further, the use of solvents enables the antimicrobial system of the present invention to be applied as a dilute solution, which forms a thin homogenous antimicrobial film. Advantageous solvents are those which readily evaporate to leave a dry film.

In a preferred embodiment, the ionic liquid or the antimicrobial system of the invention may be dispensed from a pressurised aerosol spray container.

The ionic liquids and antimicrobial systems of the present invention may advantageously be applied to a wide variety of substrates and find ability in a wide variety of industries. Thus, the use of the present invention is not particularly restricted, and the ionic liquids and antimicrobial systems may be applied to any substrate desirably disinfected, sealed and imparted with long-term antimicrobial effect whether substantially porous or non-porous in nature. As used herein, “substantially porous material” is meant to indicate one that is permeable by liquids or one that admits absorption of liquids via interstices, crevices, cracks, breaks, or other spaces between portions of substrate, which may either be closely set and minute, such as the pores in wood, or widely set, large spaces, such as in a loosely woven cloth. Examples of substantially porous materials include paper products, sponges, fiber products, woven and non-woven sheeting or fabric, plaster, wood, wood by-products, some decorative laminates, foam, bricks, stone, adhesives etc., while examples of substantially non-porous materials may include ceramics, glass, metal, polymer sheets or films and the like.

The film-forming antimicrobial system of the present invention may form an impermeable seal which not only kills microbes on the outer surface of the article, but prevents recontamination of the surface.

Antimicrobial films formed from the antimicrobial system of the present invention may desirably be removed using a film remover composition, for example when the antimicrobial activity of the film is depleted and it is desired to apply a fresh coating, or if the film has become damaged.

Suitable remover compositions for the water-insoluble, hydrophobic antimicrobial films formed from the antimicrobial system of the present invention include organic solvents and aqueous detergents.

Due at least in part to the water and abrasion resistance of the film, and also the long-term effect of the antimicrobial agents, the substrate can thus be provided with long-term antimicrobial activity, i.e., for up to at least about 48 hours, preferably for up to at least about 28 days, and more preferably for up to at least about 2 years, as can be measured according to ASTM D5590.

A further benefit of the compositions of the present invention is that they are free of environmentally hazardous metal materials (previously used in the art), such as arsenic, mercury, and lead.

As noted above, the polymers may be water soluble, and/or may comprise one or more other solvents. Water-based systems, substantially free of organic solvent, may be particularly advantageous inasmuch as the presence or use of volatile organic solvents may present safety concerns in some environments or to some users. In fact, inasmuch as the present compositions may effectively seal debris, such as mould or mould spores, to a surface they are expected to provide particular benefit to users of the compositions that suffer from allergies to the same. Allergy sufferers or others exhibiting sensitivity to mould or other microorganisms often also suffer from associated respiratory difficulties, up to and including asthma. Such individuals often exhibit sensitivity to strong odours, including perfumes, smoke, pollution, smog, cleansers, and solvents and their choices of and exposure to, such items is desirably, or even necessarily limited. Water-based compositions according to the present invention are not only free from solvent odour, but also, are substantially free of any odour thereby rendering their use by, or on substrates near such individuals, non-offensive, and thus in fact beneficial.

The abrasion resistance of the antimicrobial films of the present invention also allows for pre-coating and/or treatment of substrates which may then be sold to interested parties, for example, hospitals.

In a further aspect, the present invention provides a substrate comprising an ionic liquid or an antibacterial system as defined above.

In a further aspect, the present invention provides a disinfected substrate prepared by a method as described above.

In a further aspect, the present invention provides novel ionic liquid compositions comprising an ionic liquid having the formula:

[Cat⁺][X⁻]
wherein: [Cat⁺] is one or more cationic species comprising at least one quinolinium or isoquinolinium cationic species; and
   [X⁻] is one or more anionic species

and a metal ion selected from silver, copper, tin and zinc ions.

Preferred ionic liquids within this definition and preferred metal ions are disclosed above, and said preferred ionic liquids and metal ions are also preferred in accordance with this aspect of the invention.

In a further aspect, the present invention provides an antimicrobial system comprising:

(a) a film-enhancing composition;

(b) a viscosity controlling agent;

(c) an ionic liquid having the formula:

[Cat⁺][X⁻]
wherein: [Cat⁺] is one or more cationic species comprising at least one quinolinium or isoquinolinium cationic species; and
   [X⁻] is one or more anionic species; and

(d) optionally a metal ion selected from silver, copper, tin and zinc ions.

Preferred ionic liquids within this definition and preferred metal ions are disclosed above, and said preferred ionic liquids and metal ions are also preferred in accordance with this aspect of the invention. Similarly, the film-enhancing compositions and viscosity controlling agents described above may also be used in accordance with this aspect of the invention.

In a further aspect, the present invention also provides an antimicrobial system comprising:

(a) a film-enhancing composition;

(b) a viscosity controlling agent;

(c) an ionic liquid having the formula:

[Cat⁺][X⁻]
wherein: [Cat⁺] is one or more heterocyclic cationic species selected from: imidazolium, pyridinium, pyrazolium, thiazolium, isothiazolium, azathiazolium, oxathiazolium, oxazinium, oxazolium, oxaborolium, dithiazolium, triazolium, selenazolium, oxaphospholium, pyrrolium, borolium, furanium, thiophenium, phospholium, pentazolium, indolium, indolinium, iso-oxazolium, iso-triazolium, tetrazolium, benzofuranium, thiadiazolium, pyrimidinium, pyrazinium, pyridazinium, piperazinium, piperidinium, morpholinium, pyranium, annulenium, phthalazinium, quinazolinium, quinoxalinium, thiazinium, azaannulenium, pyrrolidinium, diazabicycloundecenium, diazabicyclononenium, diazabicyclodecenium and triazadecenium; and
   [X⁻] is one or more anionic species; and

(d) a metal salt selected from silver, copper, tin and zinc salts.

Preferred ionic liquids within this definition and preferred metal ions are disclosed above, and said preferred ionic liquids and metal ions are also preferred in accordance with this aspect of the invention. Similarly, the film-enhancing compositions and viscosity controlling agents described above may also be used in accordance with this aspect of the invention.

In a further aspect, the present invention provides a kit of parts for preparing novel ionic liquid compositions as defined above comprising:

(a) an ionic liquid having the formula:

[Cat⁺][X⁻]
wherein: [Cat⁺] is one or more cationic species comprising at least one quinolinium or isoquinolinium cationic species; and
   [X⁻] is one or more anionic species; and

(b) a metal salt selected from silver, copper, tin and zinc salts.

In a further aspect, the present invention provides a kit of parts for preparing an antimicrobial system as defined above comprising:

(a) a film-enhancing composition;

(b) a viscosity controlling agent;

(c) an ionic liquid having the formula:

[Cat⁺][X⁻]
wherein: [Cat⁺] is one or more cationic species comprising at least one quinolinium or isoquinolinium cationic species; and
   [X⁻] is one or more anionic species; and

(d) optionally a metal salt selected from silver, copper, tin and zinc salts.

In a further aspect, the present invention provides a kit of parts for preparing an antimicrobial system as described above comprising:

(a) a film-enhancing composition;

(b) a viscosity controlling agent;

(c) an ionic liquid having the formula:

[Cat⁺][X⁻]
wherein: [Cat⁺] is one or more heterocyclic cationic species selected from: imidazolium, pyridinium, pyrazolium, thiazolium, isothiazolium, azathiazolium, oxathiazolium, oxazinium, oxazolium, oxaborolium, dithiazolium, triazolium, selenazolium, oxaphospholium, pyrrolium, borolium, furanium, thiophenium, phospholium, pentazolium, indolium, indolinium, iso-oxazolium, iso-triazolium, tetrazolium, benzofuranium, thiadiazolium, pyrimidinium, pyrazinium, pyridazinium, piperazinium, piperidinium, morpholinium, pyranium, annulenium, phthalazinium, quinazolinium, quinoxalinium, thiazinium, azaannulenium, pyrrolidinium, diazabicycloundecenium, diazabicyclononenium, diazabicyclodecenium and triazadecenium; and
   [X⁻] is one or more anionic species; and

(d) a metal salt selected from silver, copper, tin and zinc salts.

It will be appreciated that although the kits of parts described above have been described with reference to the preparation of ionic liquid compositions or antimicrobial systems, the present invention also relates to the use of such kits of parts for the preparation of ionic liquid compositions and antimicrobial compositions for use as antibacterial agents.

The present invention will now be described by way of example, and with reference to the accompanying Figures, in which:

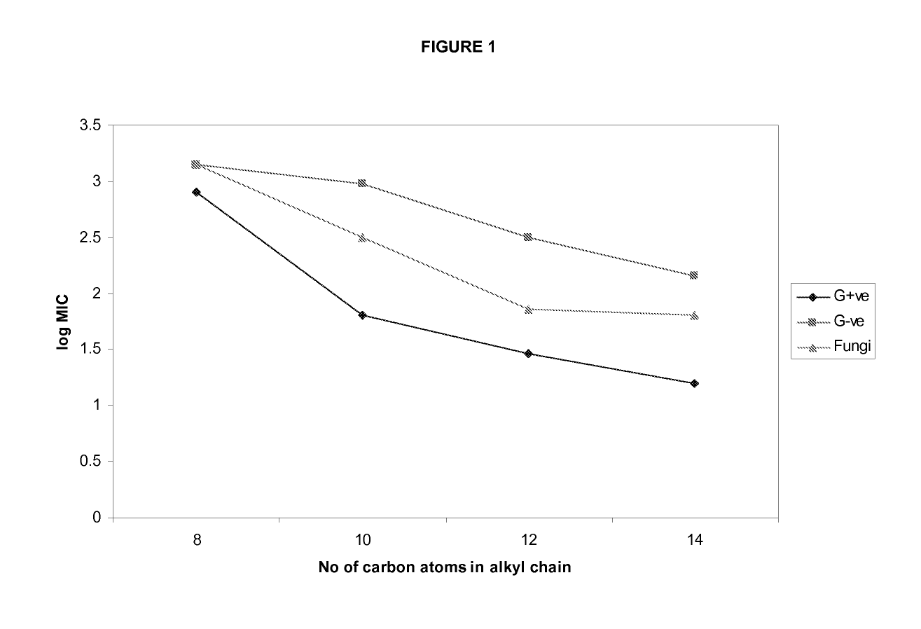

FIG. 1 is a graph showing mean minimum inhibitory concentration (MIC) values for the Gram positive cocci, Gram negative rods and fungi of the examples;

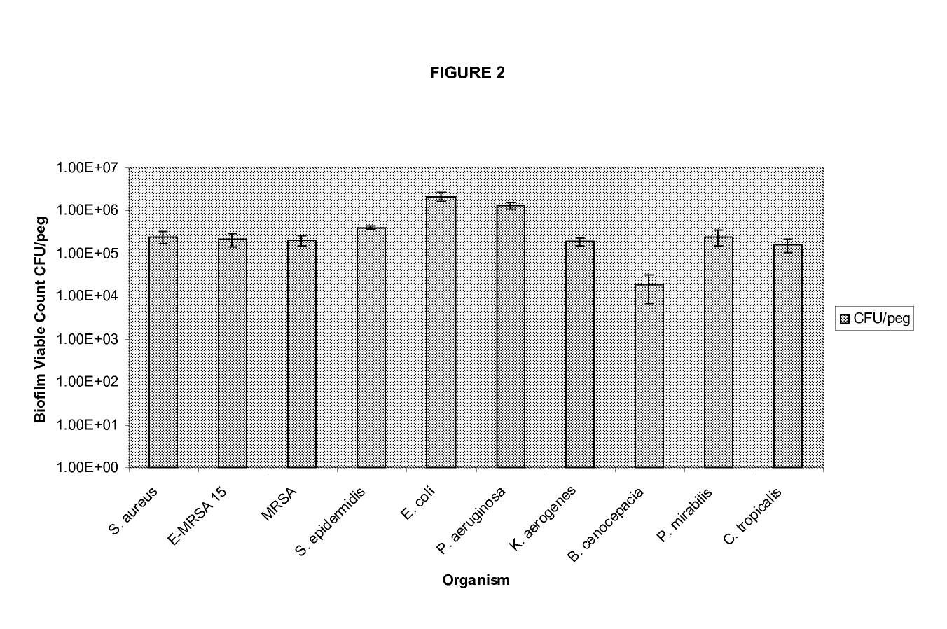

FIG. 2 shows mean 24 hour biofilm viable cell counts for each test organism grown on the Calgary Biofilm Device (vide infra). Each value is expressed as the mean of six replicates;

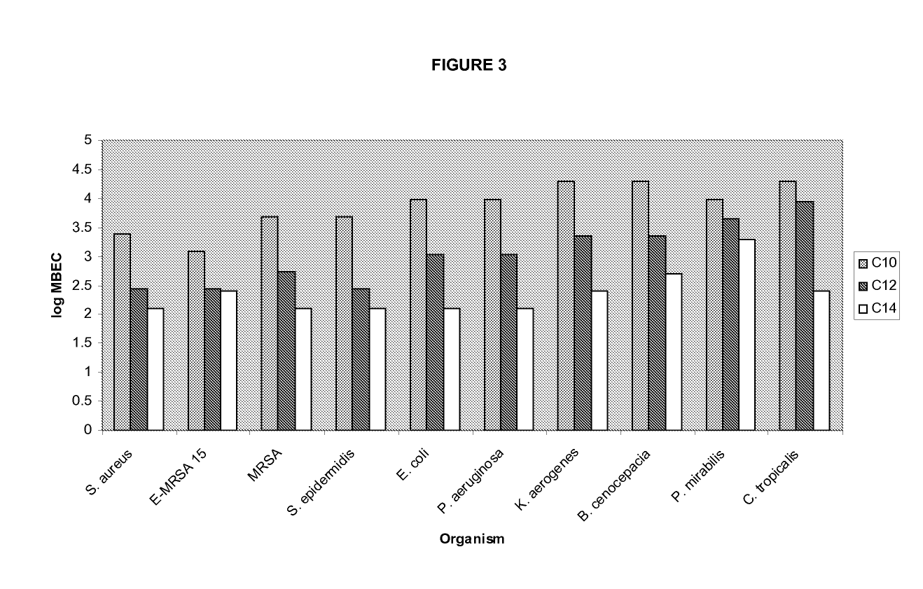

FIG. 3 is a graph showing minimum biofilm eradication concentration (MBEC) values for 1-alkyl-3-methylimidazolium chlorides with alkyl substituents of 10, 12 and 14 carbon atoms with various microorganisms;

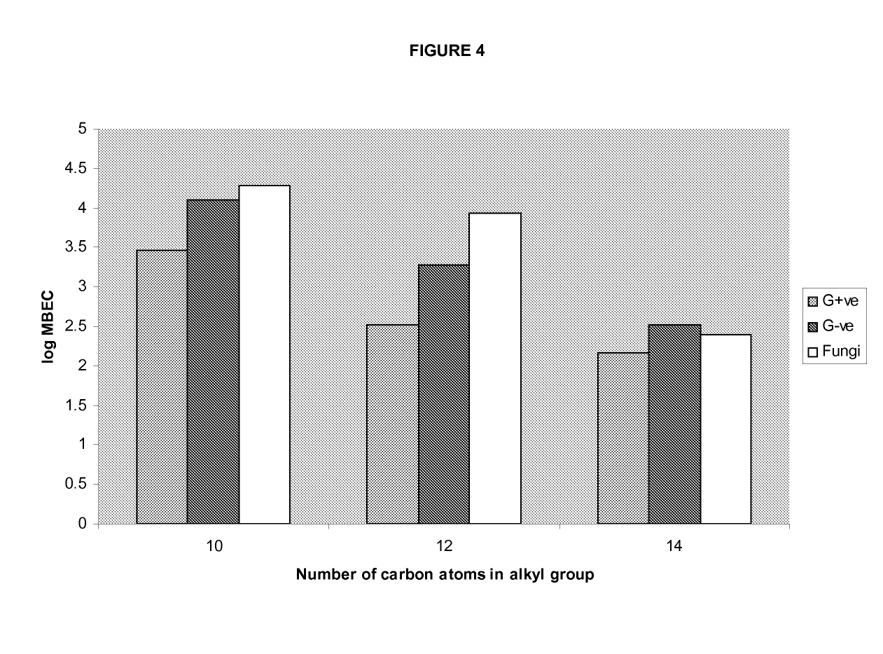

FIG. 4 shows mean minimum biofilm eradication concentration (MBEC) values for the Gram positive cocci, Gram negative rods and fungi of the examples;

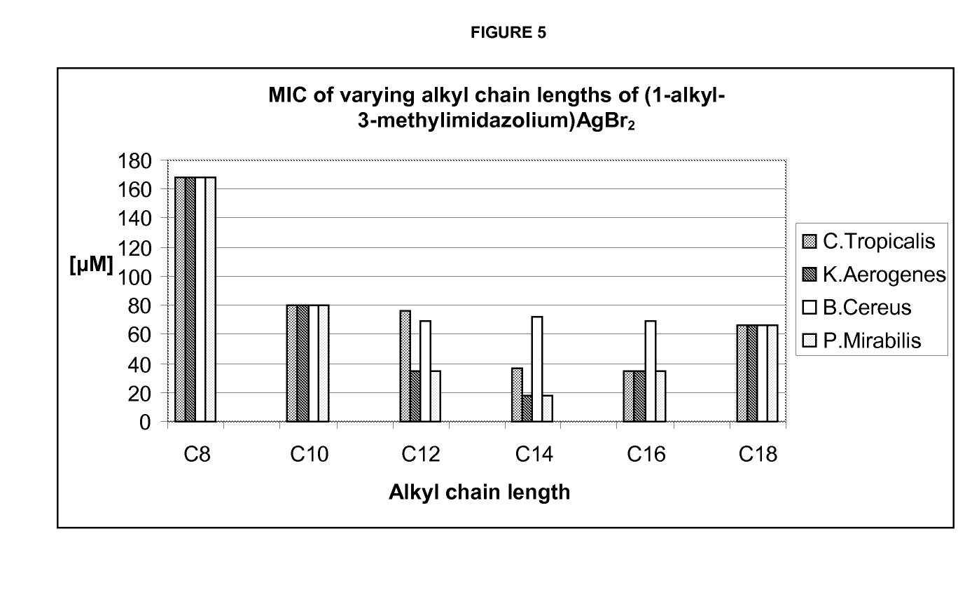

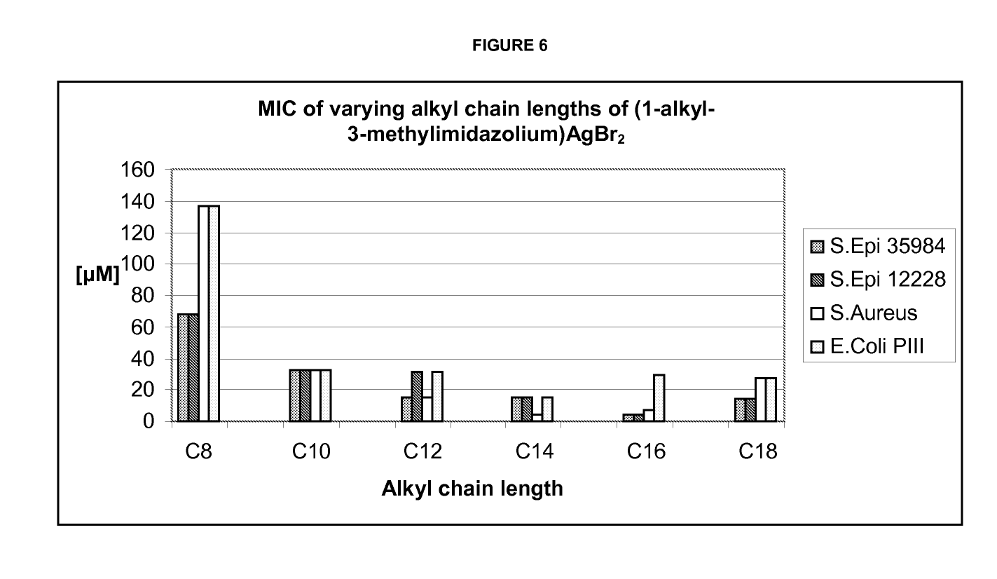

FIGS. 5 and 6 show minimum inhibitory concentration (MIC) values for [1-(C₈-C₁₈)alkyl-3-methylimidazolium][AgBr₂];

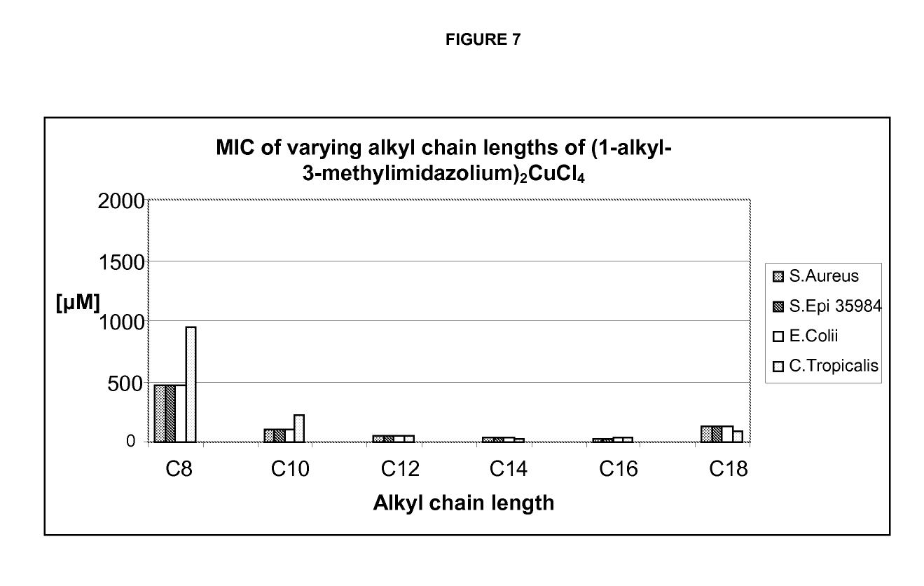

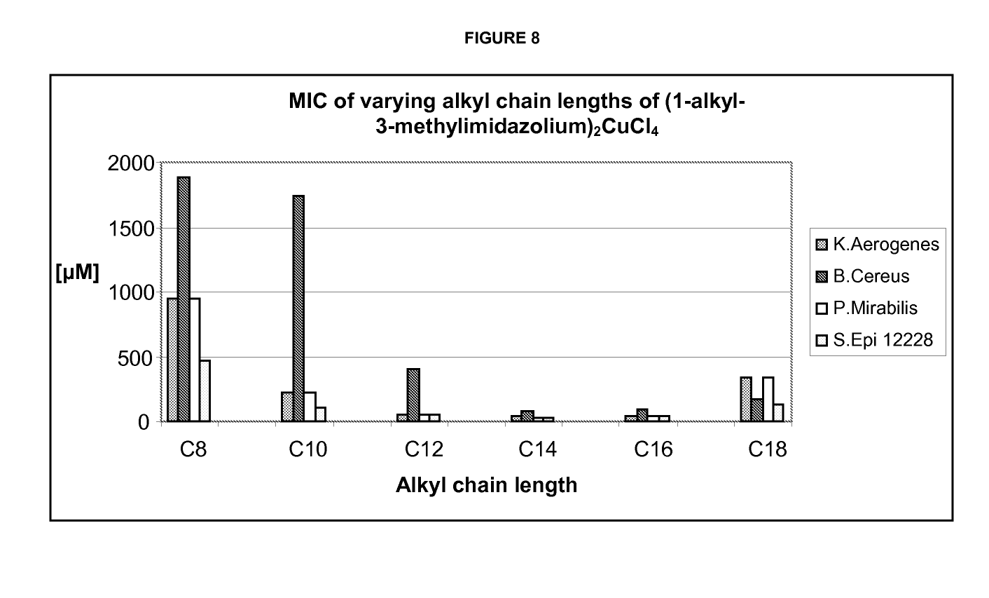

FIGS. 7 and 8 show minimum inhibitory concentration (MIC) values for [1-(C₈-C₁₈)alkyl-3-methylimidazolium]₂[CuCl₄];

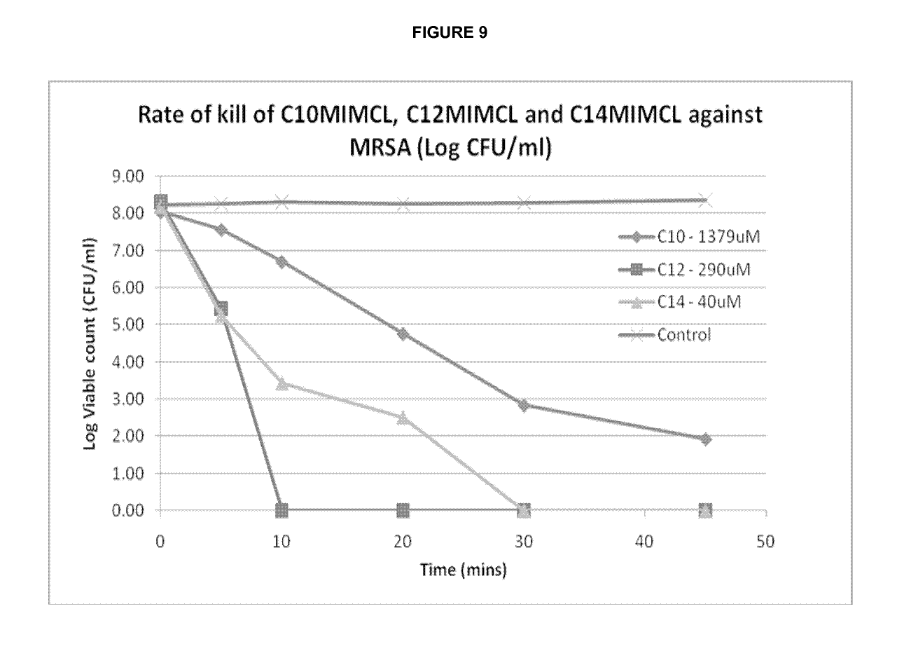

FIG. 9 is a graph showing rate of kill of MRSA for 1-decyl-3-methylimidazolium chloride (C₁₀MIMCI), 1-dodecyl-3-methylimidazolium chloride (C₁₂MIMCI), and 1-tetradecyl-3-methylimidazolium chloride (C₁₄MIMCI);

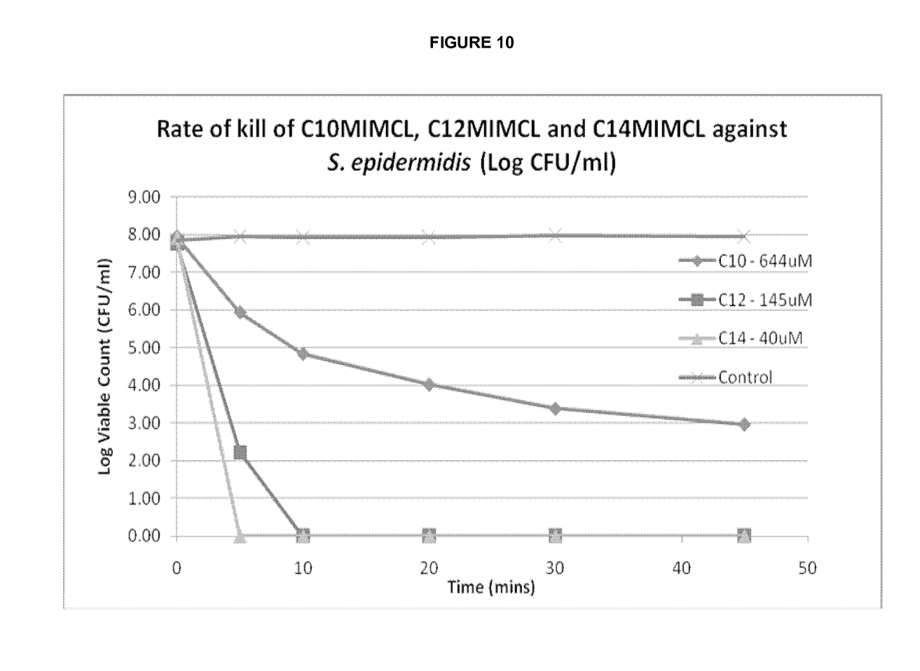

FIG. 10 is a graph showing rate of kill of S. epidermidis for 1-decyl-3-methylimidazolium chloride (C₁₀MIMCI), 1-dodecyl-3-methylimidazolium chloride (C₁₂-MIMCI), and 1-tetradecyl-3-methylimidazolium chloride (C₁₄MIMCI); and

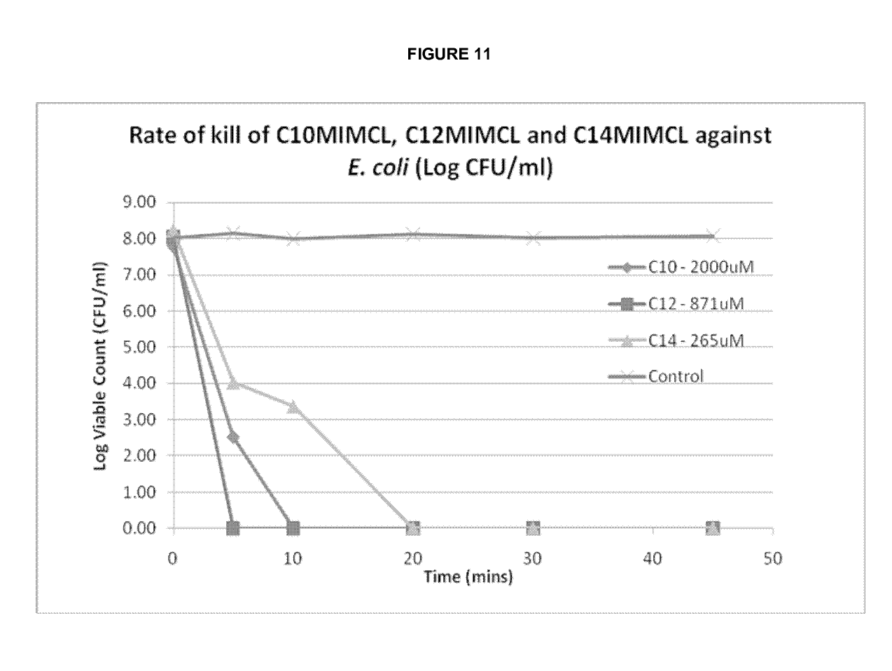

FIG. 11 is a graph showing rate of kill of E. coli for 1-decyl-3-methylimidazolium chloride (C₁₀MIMCI), 1-dodecyl-3-methylimidazolium chloride (C₁₂MIMCI), and 1-tetradecyl-3-methylimidazolium chloride (C₁₄MIMCI).

### MICROBIAL INHIBITION BY IONIC LIQUIDS

### EXAMPLE 1

Table 1 demonstrates the radius of inhibition which results from 40 μL of an imidazolium ionic liquid applied to MRSA-seeded agar culture plates. As used herein, C_{y}MIMCI, refers to 1-alkyl-3-methyl-imidazolium chloride ionic liquids, wherein the alkyl group is a straight chain alkyl group having y carbon atoms.

TABLE 1

| | Zone Of Inhibition |
|---|---|
| Compound | Radius (mm) |
| C₈MIMCl | 12 |
| C₁₀MIMCl | 17 |
| C₁₂MIMCl | 17 |
| C₁₄MIMCl | 10 |
| C₁₆MIMCl | 6 |
| C₁₈MIMCl | 10 |

### EXAMPLE 2

### MIC/MBC Determination

Tables 2 and 3 demonstrate minimum inhibitory concentrations (MIC) and minimum bactericidal concentrations (MBC) of 1-alkyl-quinolinium bromide ionic liquids and 1-alkyl-3-methyl-imidazolium chloride ionic liquids respectively, against a range of bacterial strains and the fungus C. tropicalis, wherein the 1-alkyl group is a straight chain alkyl group containing the number of carbon atoms indicated.

Broth microdilution tests were performed according to NCCLS guidelines. Serial two-fold dilutions of each imidazolium salt (from an original working solution which had been 0.22 μm sterile filtered) in Mueller-Hinton Broth (MHB) (100 μl) were prepared in 96-well micro-titre plates over the range 0.0000625-1% w/v. The inoculum to be tested was prepared by adjusting the turbidity of an actively overnight growing broth culture in MHB to an optical density at 550 nm equivalent to 1×10⁸ CFU/ml. The suspension was further diluted to provide a final inoculum density of 2×10⁵ CFU/ml in MHB as verified by total viable count. The inoculum to be tested (100 μl, 2×10⁵ CFU ml⁻¹) was added to each well of the microdilution trays which were incubated aerobically for 24 h at 37° C. Positive and negative growth controls were included in every assay (6 replicates). After determining the MICs, minimum bactericidal concentrations (MBCs) were determined by spreading 20 μl of suspension from wells showing no growth onto MHA plates, which were then incubated for 24 h and examined for 99.9% killing.

TABLE 2
MIC and MBC values of 1-alkyl-3-quinolinium bromides

| | | Alkyl Chain Length | | | | | |
|---|---|---|---|---|---|---|---|
| Organism | | 8 | 10 | 12 | 14 | 16 | 18 |
| S. epidermidis | MIC | 121.2 | 55.74 | 12.9 | 5.8 | 35.12 | 104.8 [symbol] |
| ATCC 35984 | MBC | 242 | 111.48 | 25.8 | 5.8 | 35.12 | 209.7 [symbol] |
| S. epidermidis | MIC | 121.2 | 55.74 | 12.9 | 3 | 35.12 | 104.8 [symbol] |
| ATCC 12228 | MBC | 242 | 111.48 | 25.8 | 5.8 | 35.12 | 104.8 [symbol] |
| E-MRSA 15 | MIC | 242 | 55.74 | 25.8 | 5.8 | 35.12 | 209.7 [symbol] |
| | MBC | 484 | 111.48 | 25.8 | 5.8 | 70.24 | 209.7 [symbol] |
| S. aureus | MIC | 242 | 111.5 | 25.81 | 5.8 | 70.24 | 209.7 [symbol] |
| ATCC 29213 | MBC | 484 | 111.48 | 51.6 | 5.8 | 70.24 | 419.5 [symbol] |
| E. coli | MIC | 121.2 | 55.74 | 25.81 | 11.6 | 35.12 | 104.8 [symbol] |
| NCTC 8196 | MBC | 242 | 167.22 | 38.7 | 11.6 | 70.24 | 209.7 [symbol] |
| K. aerogenes | MIC | 60 | 55.74 | 12.9 | 5.8 | 35.12 | 104.8 [symbol] |
| NCTC 7427 | MBC | 242 | 167.22 | 38.7 | 11.6 | 35.12 | 104.8 [symbol] |
| B. cereus | MIC | 484.78 | 223 | 51.63 | 23.95 | 140.4 [symbol] | 419.5 [symbol] |
| | MBC | 726 | 278.7 | 77.4 | 29.75 | 561.9 [symbol] | 839.0 [symbol] |
| P. mirabilis | MIC | 242 | 111.5 | 25.81 | 5.8 | 70.24 | 104.8 [symbol] |
| NCTC 12442 | MBC | 363 | 167.22 | 51.6 | 5.8 | 140.4 [symbol] | 209.7 [symbol] |
| P. aeruginosa | MIC | 484.78 | 223 | 51.64 | 23.95 | 70.24 | 419.5 [symbol] |
| PA01 | MBC | 605 | 446 | 90.33 | 23.95 | 280.9 [symbol] | 419.5 [symbol] |
| C. tropicalis | MIC | 242 | 55.74 | 12.9 | 3 | 35.12 | 104.8 [symbol] |
| NCTC 7393 | MBC | 363 | 111.48 | 25.8 | 3 | 70.24 | 419.5 [symbol] |

MIC and MBC values are given as micromolar concentrations (μM) [symbol]
indicates data missing or illegible when filed

TABLE 3
MIC and MBC values of 1-alkyl-3-methylimidazolium chlorides

| | | Alkyl Chain Length | | | | |
|---|---|---|---|---|---|---|
| Organism | | 6 | 8 | 10 | 12 | 14 |
| S. aureus | MIC | >1644 | 722 | 40 | 18 | 16 |
| ATCC 29213 | MBC | >1644 | >1444 | 643 | 36 | 66 |
| E-MRSA 15 | MIC | >1644 | 722 | 40 | 18 | 16 |
| | MBC | >1644 | >1444 | 321 | 73 | 66 |
| MRSA | MIC | >1644 | 1444 | 160 | 36 | 16 |
| | MBC | >1644 | >1444 | 643 | 290 | 66 |
| S. epidermidis | MIC | >1644 | 361 | 40 | 36 | 7.7 |
| ATCC 12228 | MBC | >1644 | 1444 | 644 | 145 | 33 |
| S. epidermidis | MIC | >1644 | 722 | 40 | 36 | 7.7 |
| ATCC 35984 | MBC | >1644 | 1444 | 160 | 73 | 33 |
| E. coli | MIC | >1644 | 722 | 321 | 73 | 33 |
| NCTC 8196 | MBC | >1644 | 1444 | 1287 | 73 | 33 |
| P. aeruginosa | MIC | >1644 | >1444 | >1287 | 580 | 264 |
| PA01 | MBC | >1644 | >1444 | >1287 | 1161 | 264 |
| K. aerogenes | MIC | >1644 | 1444 | 643 | 73 | 33 |
| NCTC 7427 | MBC | >1644 | >1444 | 1287 | 145 | 66 |
| B. cenocepacia | MIC | >1644 | >1444 | 1287 | 290 | 132 |
| J2315 | MBC | >1644 | >1444 | 1287 | 580 | 264 |
| P. mirabilis | MIC | >1644 | 1444 | 1287 | 580 | 264 |
| NCTC 12442 | MBC | >1644 | >1444 | 1287 | 1161 | 530 |
| C. tropicalis | MIC | >1644 | 1444 | 321 | 73 | 66 |
| NCTC 7393 | MBC | >1644 | >1444 | 321 | 73 | 132 |

MIC and MBC values are given as micromolar concentrations (μM)

### EXAMPLE 3

Table 4 demonstrates minimum inhibitory concentrations (MIC) of 1-alkyl-3-methyl-imidazolium-AgBr₂ ionic liquids against a range of bacterial strains and the fungus C. tropicalis, wherein the 1-alkyl group is a straight chain alkyl group containing the number of carbon atoms indicated. MIC values were determined according to the protocol described in Example 2.

TABLE 4
MIC values of 1-alkyl-3-methylimidazolium-AgBr₂

| | | Alkyl Chain Length | | | | | |
|---|---|---|---|---|---|---|---|
| Organism | | 8 | 10 | 12 | 14 | 16 | 18 |
| S. aureus | MIC | 136.69 | 32.68 | 15.66 | 3.76 | 7.23 | 27.83 |
| ATCC 29213 | (μM) | | | | | | |
| S. epidermidis | MIC | 68.35 | 32.68 | 31.32 | 15.03 | 3.61 | 13.91 |
| ATCC 12228 | (μM) | | | | | | |
| S. epidermidis | MIC | 68.35 | 32.68 | 15.66 | 15.03 | 3.61 | 13.91 |
| ATCC 35984 | (μM) | | | | | | |
| E. coli | MIC | 136.69 | 32.68 | 31.32 | 15.03 | 28.91 | 27.83 |
| NCTC 8196 | (μM) | | | | | | |
| K. aerogenes | MIC | 168.5 | 79.78 | 34.40 | 18.05 | 34.44 | 65.87 |
| NCTC 7427 | (μM) | | | | | | |
| B. cereus | MIC | 168.5 | 79.78 | 68.81 | 72.2 | 68.89 | 65.87 |
| | (μM) | | | | | | |
| P. mirabilis | MIC | 168.5 | 79.78 | 34.40 | 18.05 | 34.44 | 65.87 |
| NCTC 12442 | (μM) | | | | | | |
| C. tropicalis | MIC | 168.5 | 79.78 | 75.85 | 36.1 | 34.44 | 65.87 |
| NCTC 7393 | (μM) | | | | | | |

### EXAMPLE 4

Table 5 demonstrates minimum inhibitory concentrations (MIC) concentrations of (1-alkyl-3-methyl-imidazolium)₂CuCl₄ ionic liquids against a range of bacterial strains and the fungus C. tropicalis, wherein the 1-alkyl group is a straight chain alkyl group containing the number of carbon atoms indicated. MIC values were determined according to the protocol described in Example 2.

TABLE 5
MIC values of (1-alkyl-3-methylimidazolium)₂CuCl₄

| | | Alkyl Chain Length | | | | | |
|---|---|---|---|---|---|---|---|
| Organism | | 8 | 10 | 12 | 14 | 16 | 18 |
| S. aureus | MIC | 470 | 101.74 | 50.88 | 40.02 | 22.41 | 131.98 |
| ATCC 29213 | (μM) | | | | | | |
| S. epidermidis | MIC | 470 | 101.74 | 50.88 | 20.01 | 44.81 | 131.98 |
| ATCC 12228 | (μM) | | | | | | |
| S. epidermidis | MIC | 470 | 101.74 | 50.88 | 40.02 | 22.41 | 131.98 |
| ATCC 35984 | (μM) | | | | | | |
| E. coli | MIC | 470 | 101.74 | 50.88 | 40.02 | 44.81 | 131.98 |
| NCTC 8196 | (μM) | | | | | | |
| K. aerogenes | MIC | 941.6 | 218.3 | 50.88 | 40.02 | 44.81 | 338.29 |
| NCTC 7427 | (μM) | | | | | | |
| B. cereus | MIC | 1883.21 | 1746.4 | 407 | 80.06 | 89.63 | 169.15 |
| | (μM) | | | | | | |
| P. mirabilis | MIC | 941.6 | 218.3 | 50.88 | 20.01 | 44.81 | 338.29 |
| NCTC 12442 | (μM) | | | | | | |
| C. tropicalis | MIC | 941.6 | 218.3 | 50.88 | 20.01 | 44.81 | 84.57 |
| NCTC 7393 | (μM) | | | | | | |

### EXAMPLE 5

Table 6 demonstrates comparative minimum inhibitory concentrations (MIC) of ionic liquids falling outside the present invention (IL1 and IL2) and ionic liquids used according to the present invention (IL3 and IL4). MIC values were determined according to the protocol described in Example 2. The ionic liquids used are

IL1—1,2-dimethyl-3-tetradecylimidazolium bromide

IL2—N,N-dimethyl-N-(2-hydroxyethyl)-N-tetradecylammonium bromide

IL3—N-tetradecyl-6-methylquinolinium bromide

IL4—N-tetradecylisoquinolinium bromide

TABLE 6
MIC values of further ionic liquids

| | | Ionic Liquid | | | |
|---|---|---|---|---|---|
| Organism | | IL1 | IL2 | IL3 | IL4 |
| E. coli | MIC | 46.45 | 53.36 | 11.6 | 24.1 |
| NCTC 8196 | (μM) | | | | |
| S. epidermidis | MIC | 213.21 | N/A | 8.2 | 7.9 |
| ATCC 12228 | (μM) | | | | |
| P. aeruginosa | MIC | 743 | 426.9 | 23.2 | 48.2 |
| PA01 | (μM) | | | | |
| S. epidermidis | MIC | N/A | N/A | N/A | 7.9 |
| ATCC 35984 | (μM) | | | | |
| B. cereus | MIC | 371.6 | 13.3 | 2.9 | 3 |
| | (μM) | | | | |
| S. aureus | MIC | N/A | N/A | N/A | 7.9 |
| ATCC 29213 | (μM) | | | | |
| P. mirabilis | MIC | 371.6 | 426.9 | 92.9 | 192.7 |
| NCTC 12442 | (μM) | | | | |
| K. aerogenes | MIC | N/A | N/A | N/A | 7.9 |
| NCTC 7427 | (μM) | | | | |
| MRSA | MIC | 185.8 | N/A | 23.25 | 24.08 |
| | (μM) | | | | |

Biofilm Assay

### EXAMPLE 6

Tables 7 and 8 compares MIC and MBEC (minimum biofilm eradication concentration) values of 1-alkyl-quinolinium bromide ionic liquids and 1-alkyl-3-methyl-imidazolium chloride ionic liquids respectively against a range of biofilms, wherein the 1-alkyl group is a straight chain alkyl group containing the number of carbon atoms indicated. S. epidermidis ATCC 12228 is not included in the biofilm assay as it does not form biofilms.

Biofilms of each test organism were grown in the Calgary Biofilm Device (commercially available as the MBEC Assay™ for Physiology & Genetics, Innovotech Inc., Edmonton, Alberta, Canada). The device, a micro-titre plated based assay, consists of two parts; a microtitre plate containing the inoculated test medium and a polystyrene lid with 96 identical pegs on which the microbial biofilm forms under gyrorotary incubation. The biofilm assay was conducted according to the MBEC™ assay protocol as supplied by the manufacturer, with slight modifications. Inocula of each test organism were prepared in MHB as described above and adjusted to provide a final inoculum density of ˜10⁷ CFU ml⁻¹ (as confirmed by viable count). 150 μl of the inoculated media was transferred to each well of the 96-well microtitre plate and the assay plate lid, bearing 96 pegs was placed into the microtitre plate. The MBEC assay plates were placed in a gyrorotary incubator (37° C., 95% relative humidity) for 24 h to permit growth and comparison of 24 h biofilms of each test strain. Positive and negative growth controls were included in each plate (6 replicates). Initial and 24 h planktonic viable counts were measured, as were 24 h biofilm counts, expressed as CFU peg⁻¹, according to manufacturers instructions. After 24 h, the peg lid of the MBEC assay plate was gently rinsed three times. After rinsing, the peg lid of the MBEC assay was transferred to a ‘challenge’ plate. Serial dilutions of each imidazolium salt were prepared in 200 μl of MHB containing various concentrations of IL in each well. Positive growth control and sterility control were included in each assay plate. After exposure of the biofilm to the antimicrobial challenge for 24±1 h, the peg lid was removed from the challenged rinsed three times in 0.9% saline as described and transferred to a ‘recovery’ plate, each well contained MHB supplemented with neutralizers (final concentration in each well; 0.125% L-histidine, 0.125% L-cystiene, 0.25% reduced glutathione). Biofilms were dislodged into recovery media by sonication for 5 minutes and the peg lid discarded. The recovery plate is incubated overnight and visually checked after 24 h for turbidity. In addition, optical density measurements for each plate were recorded at 550 nm. Clear wells were taken as evidence of biofilm eradication and the MBEC value was assigned as the lowest concentration at which no growth was observed after 24 h incubation. Plates were incubated for a further 24 h to confirm biofilm eradication concentrations.

TABLE 7
MIC and MBEC values of 1-alkyl-3-quinolinium bromides

| | | Alkyl Chain Length | | | | | |
|---|---|---|---|---|---|---|---|
| Organism | | 8 | 10 | 12 | 14 | 16 | 18 |
| S. epidermidis | MIC | 121.2 | 55.74 | 12.9 | 5.8 | 35.12 | 104.88 |
| ATCC 35984 | MBEC | 3913 | 896 | 413.97 | 68.33 | 561.92 | 1678.08 |
| E-MRSA 15 | MIC | 242 | 55.74 | 25.8 | 5.8 | 35.12 | 209.76 |
| | MBEC | 7826 | 1792 | 827.94 | 136.66 | 1123.84 | 1678.08 |
| S. aureus | MIC | 242 | 111.5 | 25.81 | 5.8 | 70.24 | 209.76 |
| PIII | MBEC | 7826 | 1956 | 619.5 | 91.11 | 1123.84 | 3356.16 |
| E. coli | MIC | 121.2 | 55.74 | 25.81 | 11.6 | 35.12 | 104.88 |
| NCTC 8196 | MBEC | 8315 | 3584 | 827.94 | 136.66 | 561.92 | 1678.08 |
| K. aerogenes | MIC | 60 | 55.74 | 12.9 | 5.8 | 35.12 | 104.88 |
| NCTC 7427 | MBEC | 9782 | 4568 | 981.25 | 136.66 | 561.92 | 6712.32 |
| B. cereus | MIC | 484.78 | 223 | 51.63 | 23.95 | 140.48 | 419.52 |
| | MBEC | 7826 | 3584 | 774.25 | 273.32 | 1123.84 | 3356.16 |
| P. mirabilis | MIC | 242 | 111.5 | 25.81 | 5.8 | 70.24 | 104.88 |
| NCTC 12442 | MBEC | 7826 | 3584 | 1655.88 | 546.64 | 2247.68 | 3356.16 |
| P. aeruginosa | MIC | 484.78 | 223 | 51.64 | 23.95 | 70.24 | 419.52 |
| PA01 | MBEC | 8315 | 1792 | 413.97 | 136.66 | 1123.84 | 6712.32 |
| C. tropicalis | MIC | 242 | 55.74 | 12.9 | 3 | 35.12 | 104.88 |
| NCTC 7393 | MBEC | 7826 | 1792 | 413.87 | 68.33 | 561.92 | 1678.08 |

MIC and MBEC values are given as micromolar concentrations (μM)

TABLE 8
MIC and MBEC values of 1-alkyl-3-methylimidazolium chlorides

| | | Alkyl Chain Length | | | |
|---|---|---|---|---|---|
| Organism | (μM) | 8 | 10 | 12 | 14 |
| S. aureus | MIC | 722 | 40 | 18 | 16 |
| ATCC 29213 | MBEC | 2708 | 2415 | 272 | 124 |
| E-MRSA 15 | MIC | 722 | 40 | 18 | 16 |
| | MBEC | 2708 | 1207 | 272 | 248 |
| MRSA | MIC | 1444 | 160 | 36 | 16 |
| | MBEC | 21666 | 4829 | 545 | 124 |
| | | (0.5) | (0.125) | (0.016) | (0.004 |
| S. epidermidis | MIC | 722 | 40 | 36 | 7.75 |
| ATCC 35984 | MBEC | 10833 | 4829 | 272 | 124 |
| | | (0.25) | (0.125) | (0.008) | (0.004 |
| E. coli | MIC | 722 | 321 | 73 | 33 |
| PIII | MBEC | 21666 | 9659 | 1089 | 124 |
| | | | (0.25) | (0.031) | (0.004 |
| P. aeruginosa | MIC | 5416* | 2415* | 580 | 264 |
| PA01 | MBEC | 21666 | 2415 | 1089 | 496 |
| | | | (0.063) | (0.031) | (0.016 |
| K. aerogenes | MIC | 1444 | 643 | 73 | 33 |
| NCTC 7427 | MBEC | 43331 | 19318 | 2179 | 248 |
| B. cenocepacia | MIC | >1444 | 1287 | 290 | 132 |
| J2315 | MBEC | 43331 | 19318 | 2179 | 496 |
| P. mirabilis | MIC | 1444 | 1287 | 580 | 264 |
| NCTC 12442 | MBEC | 43331 | 9659 | 4357 | 1984 |
| C. tropicalis | MIC | 1444 | 321 | 73 | 66 |
| NCTC 7393 | MBEC | >43331 | 19318 | 8714 | 248 |

MIC and MBC values are given as micromolar concentrations (μM)
Numbers in parentheses indicate percent weight to volume of ionic liquid (% w/v)
in the ionic liquid composition that corresponds to the MBEC value in μM.

### EXAMPLE 7

Table 9 demonstrates minimum biofilm eradication concentration (MBEC) values obtained for the ionic liquids IL3 (N-tetradecyl-6-methylquinolinium bromide) and IL4 (N-tetradecylisoquinolinium bromide).

TABLE 9
MBEC values of further ionic liquids

Ionic Liquid

| Organism | | IL3 | IL4 |
|---|---|---|---|
| S. aureus | MBEC | | 126.9 |
| ATCC 29213 | (μM) | | |
| S. epidermidis | MBEC | 8.2 | 31.7 |
| ATCC 12228 | (μM) | | |
| S. epidermidis | MBEC | | 31.5 |
| ATCC 35984 | (μM) | | |
| K. aerogenes | MBEC | | 63.4 |
| NCTC 7427 | (μM) | | |
| B. cereus | MBEC | | 7.9 |
| | (μM) | | |

### EXAMPLE 8

Table 10 compares percent weight to volume (% w/v) and pM concentrations for the 1-(C₈-C₁₄)alkyl-3-methyl imidazolium ionic liquids exemplified.

TABLE 10
Ionic liquid concentrations in biofilm assay
Concentrations of C_{y}MIMCl (% w/v and respective concentration in μM)

| | 1.000% | 0.500% | 0.250% | 0.125% | 0.063% | 0.031% | 0.016% | 0.008% | 0.004% | 0.002% |
|---|---|---|---|---|---|---|---|---|---|---|
| C8 | 4333 | 2166 | 1083 | 5416 | 2708 | 1354 | 677 | 339 | 169 | 85 |
| C10 | 3863 | 1931 | 9659 | 4829 | 2415 | 1207 | 604 | 302 | 151 | 75 |
| C12 | 3485 | 1742 | 8714 | 4357 | 2179 | 1089 | 545 | 272 | 136 | 68 |
| C14 | 3175 | 1587 | 7938 | 3969 | 1984 | 992 | 496 | 248 | 124 | 62 |

MRSA Kill Kinetics

### EXAMPLE 9

Tables 11a-11c demonstrate the rate of bacterial cell death for MRSA, S. Epidermidis, and E. Coli planktonic cell cultures treated with 1-alkyl-3-methyl-imidazolium chloride ionic liquids in which the 1-alkyl group is decyl (C10), dodecyl (C12) and tetradecyl (C14) respectively. The kill kinetic data is provided in terms of the measured colony forming units per millilitre (CFU/mL) values, and is also provided graphically in FIGS. 9-11.

TABLE 11a
Methicillin Resistant Staphylococcus Aureus (MRSA)

| Time | C₁₀MIMCl | C₁₂MIMCl | C₁₄MIMCl | |
|---|---|---|---|---|
| Minutes | 1379 μM | 290 μM | 40 μM | Control |
| 0 | 1.10 × 10⁸ | 2.08 × 10⁸ | 1.52 × 10⁸ | 1.68 × 10⁸ |
| 5 | 3.67 × 10⁷ | 2.75 × 10⁵ | 1.78 × 10⁵ | 1.78 × 10⁸ |
| 10 | 5.00 × 10⁶ | 0.00 | 2.75 × 10³ | 1.98 × 10⁸ |
| 20 | 5.75 × 10⁴ | 0.00 | 3.33 × 10² | 1.76 × 10⁸ |
| 30 | 6.67 × 10² | 0.00 | 0.00 | 1.95 × 10⁸ |
| 45 | 8.33 × 10¹ | 0.00 | 0.00 | 2.28 × 10⁸ |

TABLE 11b
Staphylococcus Epidermidis

| Time | C₁₀MIMCl | C₁₂MIMCl | C₁₄MIMCl | |
|---|---|---|---|---|
| Minutes | 644 μM | 145 μM | 40 μM | Control |
| 0 | 8.96 × 10⁷ | 6.25 × 10⁷ | 8.67 × 10⁷ | 7.13 × 10⁷ |
| 5 | 8.58 × 10⁵ | 1.67 × 10² | 0.00 | 8.75 × 10⁷ |
| 10 | 6.92 × 10⁴ | 0.00 | 0.00 | 8.25 × 10⁷ |
| 20 | 1.06 × 10⁴ | 0.00 | 0.00 | 8.58 × 10⁷ |
| 30 | 2.42 × 10³ | 0.00 | 0.00 | 9.50 × 10⁷ |
| 45 | 9.17 × 10² | 0.00 | 0.00 | 8.75 × 10⁷ |

TABLE 11c
Escherichia Coli

| Time | C₁₀MIMCl | C₁₂MIMCl | C₁₄MIMCl | |
|---|---|---|---|---|
| Minutes | 2000 μM | 871 μM | 265 μM | Control |
| 0 | 6.25 × 10⁷ | 1.13 × 10⁸ | 1.67 × 10⁸ | 1.04 × 10⁸ |
| 5 | 3.33 × 10² | 0.00 | 1.07 × 10⁴ | 1.42 × 10⁸ |
| 10 | 0.00 | 0.00 | 2.33 × 10³ | 1.00 × 10⁸ |
| 20 | 0.00 | 0.00 | 0.00 | 1.33 × 10⁸ |
| 30 | 0.00 | 0.00 | 0.00 | 1.08 × 10⁸ |
| 45 | 0.00 | 0.00 | 0.00 | 1.17 × 10⁸ |

Film Preparation

### EXAMPLE 10

A water-soluble film composition was prepared by combining 5 parts polyvinyl alcohol polymer having a molecular weight of 180,000 Daltons (Sigma-Aldrich Chemical Company) with 95 parts water, and shaking the mixture in a warm bath for 24 hours to fully dissolve the polymer.

### EXAMPLE 11

A further film composition was prepared by dissolving polyvinyl pyrrolidone (2% solids in water—from International Specialty Products) in a 50:50 solution of isopropanol and methyl ethyl ketone.

### EXAMPLE 12

30 parts of the film composition of Example 10 were mixed with 0.2 parts of 1-octyl-3-methyl pyridinium tetrafluoroborate.

### EXAMPLE 13

6 wt % of 1-octyl-2-methyl pyridazinium tetrafluoroborate was mixed with the film composition of Example 11.

Testing

### EXAMPLE 14

(A) The composition of Example 12 was painted onto two polypropylene substrates and dried at 55° C. for 5 minutes.

(B) The composition of Example 13 was coated onto two polyethylene terephthalate substrates, dried at room temperature for 20 minutes and then at 80° C. for 10 minutes.

Control films were also produced using the compositions of Example 10 and Example 11 respectively.

Cultures of E. coli and B. subtilis were grown using known standard methods, and agar slurries comprising E. coli and B. subtilis were produced. Approximately 0.5 ml of the slurries was placed on the samples.

The samples were left for 12 hours, and after this time surviving micro-organisms were recovered via elution of the agar slurry from the test substrate into D/E Neutralizing broth, and extracted by sonication and vortexing.

Serial dilutions were prepared, and applied to agar plates, which were incubated for 48 hours at approximately 28° C.

Bacterial colony forming units were then counted.

Results

The results showed significantly less/non-detection of bacterial colonies for the samples containing the ionic liquids, when compared with the control samples, thereby demonstrating that the ionic liquids of the present invention possess good antimicrobial properties and can be successfully formulated so as to produce thin films.

Abrasion Testing

### EXAMPLE 15

Samples of films produced in accordance with Example 14 were tested for ease of removal.

A cloth was saturated with water at room temperature and rubbed over the antimicrobial film. The film required at least two strokes to be removed, and remained in solid form, i.e. did not readily dissolve.

The present application also includes the following clauses:

1. Use of an ionic liquid having the formula [Cat⁺][X⁻]
wherein [Cat⁺] is one or more heterocyclic cationic species selected from: imidazolium, pyridinium, pyrazolium, thiazolium, isothiazolium, azathiazolium, oxathiazolium, oxazinium, oxazolium, oxaborolium, dithiazolium, triazolium, selenazolium, oxaphospholium, pyrrolium, borolium, furanium, thiophenium, phospholium, pentazolium, indolium, indolinium, iso-oxazolium, iso-triazolium, tetrazolium, benzofuranium, thiadiazolium, pyrimidinium, pyrazinium, pyridazinium, piperazinium, piperidinium, morpholinium, pyranium, annulenium, phthalazinium, quinazolinium, quinoxalinium, thiazinium, azaannulenium, pyrrolidinium, diazabicycloundecenium, diazabicyclononenium, diazabicyclodecenium and triazadecenium; and
   [X⁻] is one or more anionic species,
      and further comprising a metal ion selected from silver, copper, tin and zinc ions, as an antibacterial agent.

2. The use according to Clause 1, wherein the metal ion is selected from Ag⁺, Cu⁺, Cu²⁺, Sn²⁺ and Zn²⁺ ions; more preferably Ag⁺, Cu⁺ and Cu²⁺ ions; and most preferably Ag⁺ and Cu²⁺ ions.

3. The use according to Clause 2, wherein the metal ion is in the form of a complex; more preferably a metal halide complex; more preferably a metal chloride or bromide complex; and most preferably a complex having the formula [Ag⁺][Br⁻]₂ or the formula [Cu²⁺][Cl⁻]₄.

4. The use according to any of Clauses 1 to 3, wherein the one or more heterocyclic cationic species are selected from:

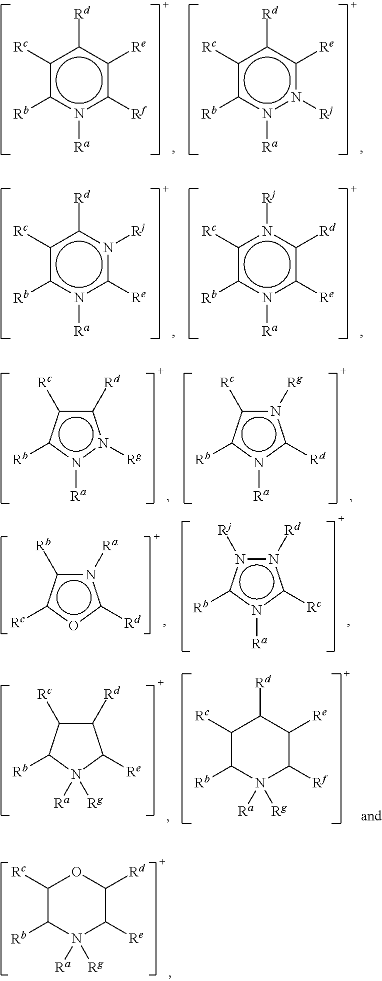

wherein: R^{a}, R^{b}, R^{c}, R^{d}, R^{e}, R^{f}, R^{g}, R^{h}, Rⁱ and R^{j} can be the same or different, and are each independently selected from hydrogen, a C₁ to C₄₀, straight chain or branched alkyl group, a C₃ to C₈ cycloalkyl group, or a C₆ to C₁₀ aryl group, wherein said alkyl, cycloalkyl or aryl groups are unsubstituted or may be substituted by one to three groups selected from: C₁ to C₆ alkoxy, C₆ to C₁₀ aryl, CN, OH, NO₂, C₇ to C₃₀ aralkyl and C₇ to C₃₀ alkaryl, or any two of R^{b}, R^{c}, R^{d}, R^{e}, R^{f}, R^{h} and Rⁱ attached to adjacent carbon atoms form a methylene chain —(CH₂)_{q}— wherein q is from 8 to 20, and wherein R^{j} may be absent.

5. The use according to Clause 4, wherein the one or more cationic species are selected from:

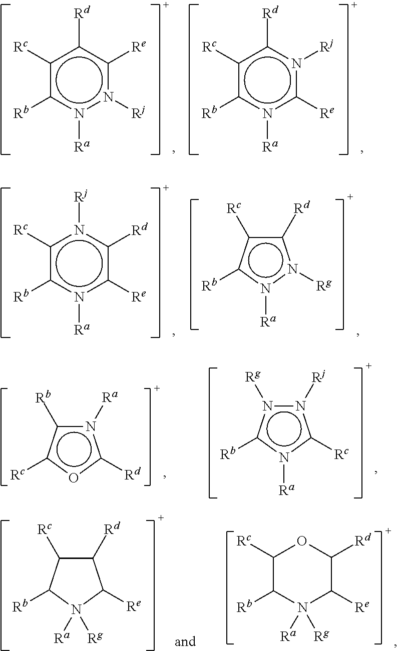

wherein: R^{a}, R^{b}, R^{c}, R^{d}, R^{e}, R^{f}, R^{g}, R^{h}, Rⁱ and R^{j} are as defined in Clause 4.

6. The use according to Clause 5, wherein the one or more further cationic species are selected from:

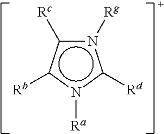

wherein: R^{a}, R^{b}, R^{c}, R^{d}, R^{g} are as defined in Clause 4.

7. The use according to any of Clauses 4 to 6, wherein R^{a}, R^{g} and R^{j} are independently selected, where present, from C₁ to C₃₀ linear or branched alkyl, and one of R^{a}, R^{g} and R^{j} may also be hydrogen.

8. The use according to Clause 7, wherein R^{a} is selected from C₂ to C₂₀ linear or branched alkyl; more preferably C₄ to C₁₈ linear or branched alkyl; still more preferably C₈ to C₁₈ linear or branched alkyl; and most preferably C₈ to C₁₄ linear or branched alkyl.

9. The use according to Clause 7 or Clause 8, wherein R^{g} and R^{j} are independently selected, where present, from C₁ to C₁₀ linear or branched alkyl; more preferably C₁ to C₅ linear or branched alkyl; and most preferably a methyl group.

10. The use according to Clause 7, wherein one of R^{a}, R^{g} and R^{j} is selected from ethyl, butyl, hexyl, octyl, nonyl, decyl, undecyl, dodecyl, tridecyl, tetradecyl, pentadecyl, hexadecyl, heptadecyl and octadecyl.

11. The use according to any of Clauses 4 to 6, wherein R^{a}, R^{g} and R^{j} are each independently selected, where present, from C₁ to C₃₀ linear or branched alkyl, and C₁ to C₁₅ alkoxyalkyl.

12. The use according to any of the preceding clauses, wherein [X⁻] is one or more anionic species selected from: [BF₄]⁻, [PF₆]⁻, [SbF₆]⁻, [F]⁻, [Cl]⁻, [Br]⁻, [I]⁻, [NO₃]⁻, [NO₂]⁻, [H₂PO₄]⁻, [HPO₄]²⁻, [R^{x}₂PO₄]²⁻, [R^{x}₃PF₃]⁻, [R^{x}₂P(O)O]⁻[HSO₃]⁻, [HSO₄]⁻, [R^{x}SO₃]⁻, [R^{x}SO₄]⁻, [SO₄]²⁻, [H₃CO(CH₂)₂O(CH₂)OSO₃]⁻, [BBDB]⁻, [BOB]⁻, [(CF₃SO₂)₃C]⁻, [Co(CO₄)]⁻, [(CN)₂N]⁻, [(CF₃)₂N]⁻, [(R^{x}SO₂)₂N]⁻, [SCN]⁻, [H₃C(OCH₂CH₂)ₙOSO₃]⁻, [R^{x}O₂CCH₂CH(CO₂R^{x})SO₃]⁻, [R^{x}CO₂]⁻, lactate, and docusate; wherein each R^{x} is independently selected from (C₁-C₂₀)alkyl, (C₆-C₁₀)aryl, and (C₁-C₁₀)alkyl(C₆-C₁₀)aryl; and further wherein said alkyl, aryl and alkylaryl groups may comprise one or more substituents independently selected from F, Cl and I.

13. The use according to Clause 12 wherein [X⁻] is selected from [Cl]⁻, [Br]⁻, [I]⁻, [H₂PO₄]⁻, [HPO₄]²⁻, [R^{x}PO₄ ⁻, [R^{x}₂P(O)O⁻], [R^{x}SO₃]⁻, [CH₃SO₃]⁻, [R^{x}SO₄]⁻, [CH₃SO₄]⁻, [C₂H₅SO₄]⁻, [SO₄]²⁻, [R^{x}O₂CCH₂CH(CO₂R^{x})SO₃]⁻, [R^{x}CO₂]⁻, [C₆H₄CO₂]⁻, lactate and docusate; wherein R^{x} is as defined in Clause 12.

14. The use according to Clause 13 wherein [X⁻] is selected from [Cl]⁻, [Br]⁻, and [I]⁻.

15. The use according to Clause 12 wherein [X⁻] is selected from [BF₄]⁻, [PF₆]⁻, [BBDB]⁻, [BOB]⁻, [N(CF₃)₂]⁻, [(CF₃SO₂)₂N]⁻, [(CF₃SO₂)₃C]⁻, [(C₂F₅)₃PF₃]⁻, [(C₃F₇)₃PF₃]⁻, [(C₂F₅)₂P(O)O]⁻, [SbF₆]—, [Co(CO)₄]⁻, [NO₃]⁻, [NO₂]⁻, [CF₃SO₃]⁻, [CH₃SO₃]⁻, [C₈H₁₇OSO₃]⁻, and tosylate.

16. Use of ionic liquids comprising a species having the formula:

[Cat⁺][M⁺][X⁻]
wherein: [Cat⁺] is one or more heterocyclic cationic species, as defined in any of Clauses 1 and 4 to 11;
   [M⁺] is one or more metal ions, as defined in any of Clauses 1 to 3; and
   [X⁻] is one or more anionic species, as defined in any of Clauses 1 and 12 to 15,
      as antibacterial agents.

17. The use according to Clause 16, wherein the ionic liquid comprises a species having the formula C₍₈₋₁₈₎MIMAgBr₂ or (C₍₈₋₁₈₎MIM)₂CuCl₄; and more preferably a species having the formula C₍₁₂₋₁₆₎MIMAgBr₂ ^{or} (C₍₁₂₋₁₆)MIM)₂CuCl₄.

18. The use according to any of the preceding clauses, wherein the ionic liquid is used in the form of an antimicrobial system comprising:

(a) a film-enhancing composition;

(b) a viscosity controlling agent; and

(c) the ionic liquid, as defined in any of Clauses 1 to 17.

19. The use according to Clause 18, wherein the ionic liquid is present in an amount from 0.001 wt % to 10 wt %; more preferably wherein the ionic liquid is present in an amount of from 0.005 wt % to 7 wt %; and most preferably wherein the ionic liquid is present in an amount of from 0.01 wt % to 0.5 wt %.

20. The use according to Clause 18 or Clause 19, wherein the viscosity controlling agent comprises one or more solvents and/or thickening agents.

21. The use according to any of Clauses 18 to 20, wherein the film-enhancing composition comprises one or more film-enhancing components selected from wetting agents, dispersing aids, surfactants, pigments, defoaming agents, coalescing agents, fillers, reinforcing agents, adhesion promoters, plasticizers, flow control agents, antioxidants, UV stabilizers, polymers or combinations of these.

22. The use according to any of Clauses 18 to 21, wherein the film-enhancing composition is present in an amount ranging from 1 wt % to 90 wt %; more preferably 10 wt % to 80 wt %; and most preferably 15 wt % to 75 wt %.

23. The use according to any of Clauses 18 to 22, wherein the film-enhancing composition comprises a film-forming polymer.

24. The use according to Clause 23, wherein the polymer is hydrophobic and/or water-insoluble.

25. The use according to Clause 23, wherein the polymer is substantially nonionic or cationic.

26. The use according to Clause 23, wherein the polymer is substantially anionic.

27. The use according to Clause 23, wherein the polymer is water-soluble.

28. The use according to any of Clauses 18 to 27, wherein the antimicrobial system further includes an optical reporter, e.g., a fluorophore or an optical brightening agent that enables detection of the composition on a surface by suitable detection devices such as irradiation by an ultraviolet or visible light source.

29. A method of disinfecting a surface which comprises applying to the surface an ionic liquid as defined in any of Clauses 1 to 17, or an antimicrobial system as defined in any of Clauses 18 to 28.

30. A method according to Clause 29, wherein the ionic liquid or the antimicrobial system is applied as an aerosol from a pressurized aerosol spray canister.

31. A method according to Clause 29 or Clause 30 wherein the antimicrobial system, after application, is dried.

32. A substrate comprising an ionic liquid as defined in any of Clauses 1 to 17.

33. A substrate comprising an antibacterial system as defined in any of Clauses 18 to 28.

34. A substrate prepared by a method according to any of Clauses 29 to 31.

35. An antimicrobial system comprising:

(a) a film-enhancing composition;

(b) a viscosity controlling agent;

(c) an ionic liquid having the formula:

[Cat⁺][X⁻]
wherein: [Cat⁺] is one or more heterocyclic cationic species selected from:

imidazolium, pyridinium, pyrazolium, thiazolium, isothiazolium, azathiazolium, oxathiazolium, oxazinium, oxazolium, oxaborolium, dithiazolium, triazolium, selenazolium, oxaphospholium, pyrrolium, borolium, furanium, thiophenium, phospholium, pentazolium, indolium, indolinium, iso-oxazolium, iso-triazolium, tetrazolium, benzofuranium, thiadiazolium, pyrimidinium, pyrazinium, pyridazinium, piperazinium, piperidinium, morpholinium, pyranium, annulenium, phthalazinium, quinazolinium, quinoxalinium, thiazinium, azaannulenium, pyrrolidinium, diazabicycloundecenium, diazabicyclononenium, diazabicyclodecenium and triazadecenium; and
[X] is one or more anionic species; and

(d) a metal ion selected from silver, copper, tin and zinc ions.

36. An antimicrobial system according to Clause 35, wherein the metal ion is as defined in Clause 2 or Clause 3.

37. An antimicrobial system according to Clause 35 or Clause 36, wherein [Cat⁺] is one or more heterocyclic cationic species as defined in any of Clauses 4 to 11.

38. An antimicrobial system according to any of Clauses 35 to 37, wherein [X⁻] is one or more anionic species as defined in any of Clauses 12 to 15.

39. A kit of parts for preparing an antimicrobial system according to any of Clauses 35 to 38 comprising:

(a) a film-enhancing composition;

(b) a viscosity controlling agent;

(c) an ionic liquid having the formula:

[Cat⁺][X⁻]
wherein: [Cat⁺] is one or more heterocyclic cationic species selected from:

imidazolium, pyridinium, pyrazolium, thiazolium, isothiazolium, azathiazolium, oxathiazolium, oxazinium, oxazolium, oxaborolium, dithiazolium, triazolium, selenazolium, oxaphospholium, pyrrolium, borolium, furanium, thiophenium, phospholium, pentazolium, indolium, indolinium, iso-oxazolium, iso-triazolium, tetrazolium, benzofuranium, thiadiazolium, pyrimidinium, pyrazinium, pyridazinium, piperazinium, piperidinium, morpholinium, pyranium, annuleniurn, phthalazinium, quinazolinium, quinoxalinium, thiazinium, azaannulenium, pyrrolidinium, diazabicycloundecenium, diazabicyclononenium, diazabicyclodecenium and triazadecenium; and
[X⁻] is one or more anionic species; and

(d) a metal ion selected from silver, copper, tin and zinc ions.

1.-47. (canceled)

48. An antimicrobial composition comprising:
an ionic liquid having a formula:
[Cat⁺][M⁺][X⁻]
wherein: [Cat⁺] is at least one cationic species as defined below;
[M⁺] is at least one metal ion selected from a group consisting of: silver, copper, tin and zinc ions; and
[X⁻] is at least one anionic species,
and wherein [Cat⁺] comprises at least one cationic species selected from a group consisting of: 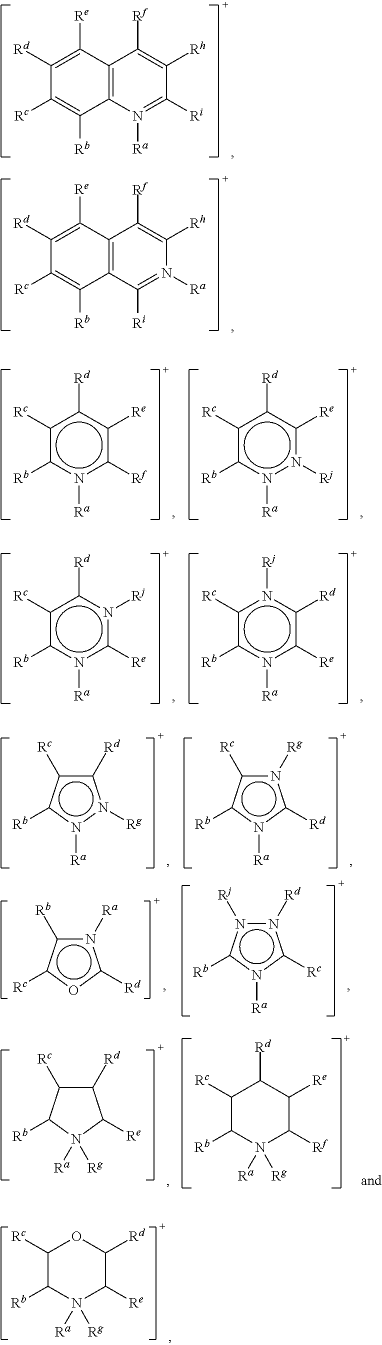
wherein: R^{b}, R^{c}, R^{d}, R^{e}, R^{f}, R^{g}, R^{h}, Rⁱ and R^{j} can be the same or different, and are each independently selected from a group consisting of hydrogen, a C₁ to C₄₀ linear or branched alkyl group, a C₃ to C₈ cycloalkyl group, or a C₆ to C₁₀ aryl group, or any two of R^{b}, R^{c}, R^{d}, R^{e}, R^{f}, R^{h} and Rⁱ attached to adjacent carbon atoms form a methylene chain —(CH₂)_{q}— wherein q is from 8 to 20, wherein said alkyl, cycloalkyl or aryl groups, or said methylene chain, are unsubstituted or substituted by one to three groups selected from a group consisting of: C₁ to C₆ alkoxy, C₆ to C₁₀ aryl, CN, OH, NO₂, C₇ to C₃₀ aralkyl and C₇ to C₃₀ alkaryl; and
wherein R^{a} is selected from a group consisting of: decyl, undecyl, dodecyl, tridecyl, tetradecyl, pentadecyl, hexadecyl, heptadecyl and octadecyl; and
wherein R^{a} and R^{g} are both present one of R^{a} and R^{g} is selected from a group consisting of: decyl, undecyl, dodecyl, tridecyl, tetradecyl, pentadecyl, hexadecyl, heptadecyl and octadecyl.

49. The antimicrobial composition of claim 48, wherein [Cat⁺] is selected from a group consisting of: 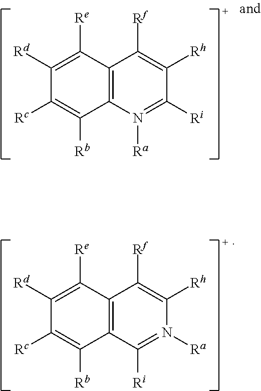
and further wherein R^{b}, R^{c}, R^{d}, R^{e}, R^{f}, R^{h} and Rⁱ are each independently selected from a group consisting of: hydrogen, and a C₁ to C₃₀ linear or branched alkyl group.

50. The antimicrobial composition of claim 49, wherein one of R^{b}, R^{c}, R^{d}, R^{e}, R^{f}, R^{h} and Rⁱ is a C₁ to C₃₀ linear or branched alkyl group, and the remainder of R^{b}, R^{c}, R^{d}, R^{e}, R^{f}, R^{h} and Rⁱ are each hydrogen.

51. The antimicrobial composition of claim 48, wherein [Cat⁺] is selected from a group consisting of: 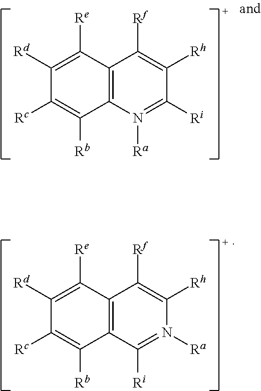
and further wherein R^{d} is a C₁ to C₃₀ linear or branched alkyl group or hydrogen, and R^{b}, R^{c}, R^{e}, R^{f}, R^{h} and Rⁱ are each hydrogen.

52. The antimicrobial composition of claim 48, wherein [Cat⁺] is one or more cationic species comprising at least one quinolinium or isoquinolinium cationic species selected from a group consisting of: N-octylquinolinium, N-decylquinolinium, N-dodecylquinolinium, N-tetradecylquinolinium, N-octylisoquinolinium, N-decylisoquinolinium, N-dodecylisoquinolinium, and N-tetradecylisoquinolinium.

53. The antimicrobial composition of claim 49, wherein [Cat⁺] is one or more cationic species comprising at least one quinolinium or isoquinolinium cationic species selected from a group consisting of: N-octyl-6-methylquinolinium, N-decyl-6-methylquinolinium, N-dodecyl-6-methyl-quinolinium, N-tetradecyl-6-methylquinolinium, N-octyl-6-methylisoquinolinium, N-decyl-6-methylisoquinolinium, N-dodecyl-6-methylisoquinolinium, and N-tetradecyl-6-methylisoquinolinium.

54. The antimicrobial composition of claim 48, wherein [Cat⁺] is selected from a group consisting of: 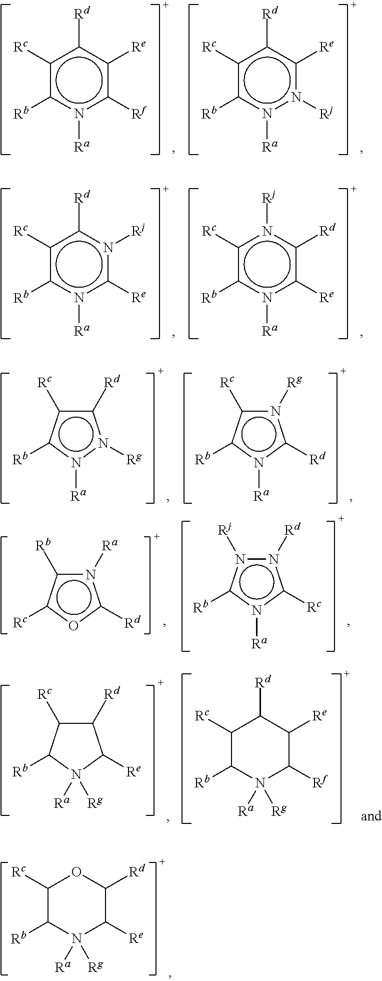
and further wherein R^{g} and R^{j} when present, are independently selected from a group consisting of: C₁ to C₃₀ linear or branched alkyl, and one of R^{g} and R^{j} is hydrogen.

55. The antimicrobial composition of claim 54, wherein R^{g} and R^{j} are selected from a group consisting of: C₁ to C₁₀ linear or branched alkyl, C₁ to C₅ linear or branched alkyl, and a methyl group.

56. The antimicrobial composition of claim 54, wherein R^{g} and R^{j} is selected from a group consisting of: ethyl, butyl, hexyl, octyl, nonyl, decyl, undecyl, dodecyl, tridecyl, tetradecyl, pentadecyl, hexadecyl, heptadecyl and octadecyl.

57. The antimicrobial composition of claim 48, wherein [X⁻] is one or more anionic species selected from a group consisting of: [BF₄]⁻, [PF₆]⁻, [SbF₆]⁻, [F]⁻, [Cl]⁻, [Br]⁻, [I]⁻, [NO₃]⁻, [NO₂]⁻, [H₂PO₄]⁻, [HPO₄]²⁻, [R^{x}₂PO₄]⁻, [R^{x}PO₄]²⁻, [R^{x}₃PF₃]⁻, [R^{x}₂P(O)O]⁻[HSO₃]⁻, [HSO₄]⁻, [R^{x}SO₃]⁻, [R^{x}SO₄]⁻, [SO₄]²⁻, [H₃CO(CH₂)₂O(CH₂)OSO₃]⁻, [BBDB]⁻, [BOB]⁻, [(CF₃SO₂)₃C]⁻, [Co(CO₄)]⁻, [(CN)₂N]⁻, [(CF₃)₂N]⁻, [(R^{x}SO₂)₂N]⁻, ]SCN]⁻, [H₃C(OCH₂CH₂)ₙOSO₃]⁻, [R^{x}O₂CCH₂CH(CO₂R^{x})SO₃]⁻, [R^{x}CO₂]⁻, lactate, and docustate; wherein each R^{x} is independently selected from a group consisting of: (C₁-C₂₀)alkyl, (C₆-C₁₀)aryl, and (C₁-C₁₀)alkyl(C₆-C₁₀)aryl.

58. The antimicrobial composition of claim 57, wherein [X⁻] is selected from a group consisting of: [Cl]⁻, [Br]⁻, [I]⁻, [H₂PO₄]⁻, [HPO₄]²⁻, [R^{x}₂PO₄]⁻, [R^{x}₂P(O)O⁻], [R^{x}SO₃]⁻, [CH₃SO₃]⁻, [R^{x}SO₄]⁻, [CH₃SO₄]⁻, [C₂H₅SO₄]⁻, [SO₄]²⁻, [R^{x}O₂CCH₂CH(CO₂R^{x})SO₃]⁻, [R^{x}CO₂]⁻, [C₆H₄CO₂]⁻, lactate and docusate, wherein each R^{x} is independently selected from a group consisting of: (C₁-C₂₀)alkyl, (C₆-C₁₀)aryl, and (C₁-C₁₀)alkyl(C₆-C₁₀)aryl.

59. The antimicrobial composition of claim 58, wherein [X⁻] is selected from a group consisting of: [Cl]⁻, [Br]⁻, and [I]⁻.

60. The antimicrobial composition of claim 57, wherein [X⁻] is selected from a group consisting of: [BF₄]⁻, [PF₆]⁻,[BBDB]⁻, [BOB]⁻, [N(CF₃)₂]⁻, [(CF₃SO₂)₂N]⁻, [(CF₃SO₂)₃C]⁻, [(C₂F₅)₃PF₃]⁻, [(C₃F₇)₃PF₃]⁻, [(C₂F₅)₂P(O)O]⁻, [SbF₆]—, [Co(CO)₄]⁻, [NO₃]⁻, [NO₂]⁻, [CF₃SO₃]⁻, [CH₃SO₃]⁻, [C₈H₁₇OSO₃]⁻, and tosylate.

61. The antimicrobial composition of claim 48, wherein the metal ion is selected from a group consisting of: Ag⁺, Cu⁺, Cu²⁺, Sn²⁺ and Zn²⁺ ions.

62. The antimicrobial composition of claim 61, wherein the metal ion is selected from a group consisting of: Ag⁺, Cu⁺ and Cu²⁺ ions.

63. The antimicrobial composition of claim 62, wherein the metal and anion have the formula [Ag⁺][Br⁻]₂ or the formula [Cu²⁺][Cl⁻]₄.

64. An antimicrobial system, comprising:
(a) a film enhancing composition;
(b) a viscosity controlling agent; and
(c) the ionic liquid defined in claim 48.

65. The antimicrobial system of claim 64, wherein the ionic liquid is present in an amount of from about 0.01 wt % to about 0.5 wt %.

66. The antimicrobial system of claim 64, wherein the viscosity controlling agent comprises at least one member of a group comprising: solvents and/or thickening agents.

67. The antimicrobial system of claim 64, wherein the film-enhancing composition comprises at least one member of a group comprising: wetting agents, dispersing aids, surfactants, pigments, defoaming agents, coalescing agents, fillers, reinforcing agents, adhesion promoters, plasticizers, flow control agents, antioxidants, UV stabilizers, and polymers.

68. The antimicrobial system of claim 64, wherein the film enhancing composition comprises a film-forming polymer.

69. The antimicrobial system of claim 68, wherein the polymer is hydrophobic and/or water-insoluble.

70. The antimicrobial system of claim 68, wherein the polymer is substantially nonionic or cationic.

71. The antimicrobial system of claim 68, wherein the polymer is substantially anionic.

72. The antimicrobial system of claim 68, wherein the polymer is water-soluble.

73. A method for disinfecting a surface which comprises applying to the surface the antimicrobial composition as defined in claim 48.

74. A substrate comprising the antimicrobial composition as defined in claim 48.

75. A substrate comprising the antimicrobial system as defined in claim 64.

76. A substrate prepared by applying the antimicrobial composition, as defined in claim 48, to a surface.

77. A kit, comprising:
(a) a film enhancing composition;
(b) a viscosity controlling agent; and
(c) an antimicrobial composition as defined in claim 48.
